SEQUENCE LISTING (1 sequence)

SEQ ID NO: 1 - protein, 22 aa, Homo sapiens
LLPKNYHLENEVARLKKLVGER

(19) United States
(12) Patent Application Publication
(10) Pub. No.: US 2025/0339557 A1
(43) Pub. Date: Nov. 6, 2025

(54) CRISPR-BASED MODULAR TOOL FOR THE SPECIFIC INTRODUCTION OF EPIGENETIC MODIFICATIONS AT TARGET LOCI

(71) Applicant: EUROPEAN MOLECULAR BIOLOGY LABORATORY, Heidelberg (DE)

(72) Inventors: Jamie HACKETT, Rome (IT); Cristina POLICARPI, Rome (IT)

(21) Appl. No.: 18/875,915

(22) PCT Filed: Jun. 24, 2023
(86) PCT No.: PCT/EP2023/067209

§ 371 Date: Dec. 17, 2024

(30) Foreign Application Priority Data
Jun. 24, 2022 (EP) 22181034.4

(51) Int. Cl. A61K 48/00; C07K 16/14; C12N 9/22; C12N 15/11; G01N 33/50

(52) U.S. Cl. CPC A61K 48/005; C07K 16/14; C12N 9/226; C12N 15/111; G01N 33/5023; C07K 2317/622; C07K 2319/80; C12N 2310/20

(57) ABSTRACT

The present invention relates to a complex comprising i) a catalytically inactive site-specific nuclease linked to ii) an array of between two and ten, preferably three to seven effector domains each having a specific chromatin modifying activity, such as, for example, a specific DNA methylation activity, a histone methylation activity, a specific histone acetylation or ubiquitination activity, and/or a specific chromatin demethylation/deacetylation activity, wherein the effector domains are each separated by a linker providing sufficient distance between the domains and the nuclease in order not to substantially interfere with their specific chromatin modifying activities, and the binding of the site-specific nuclease, as well as respective methods involving the complex and use of the complex.

The present invention relates to a complex comprising i) a catalytically inactive site-specific nuclease linked to ii) an array of between two and ten, preferably three to seven effector domains each having a specific chromatin modifying activity, such as, for example, a specific DNA methylation activity, a histone methylation activity, a specific histone acetylation or ubiquitination activity, and/or a specific chromatin demethylation/deacetylation activity, wherein the effector domains are each separated by a linker providing sufficient distance between the domains and the nuclease in order not to substantially interfere with their specific chromatin modifying activities, and the binding of the site-specific nuclease, as well as respective methods involving the complex and use of the complex.

### BACKGROUND OF THE INVENTION

The coordinated regulation of transcription is essential for almost all biological processes, ranging from development, to homeostasis, to disease. Understanding the nature, impact, and context-dependency of the molecular mechanisms that orchestrate gene expression is thus a central goal of modern biology.

Regulation of eukaryotic transcription is guided by a complex interplay between transcription factors (TF), cis regulatory elements, and epigenetic mechanisms. Such epigenetic systems are defined as the assemblage of sequence-independent regulatory molecules, either heritable or otherwise, that impact chromatin architecture, genome function, and transcriptional activity.

Most prominently, the so-called epigenome is characterized by posttranslational histone modifications and DNA methylation.

A loss of genes or functions that are involved in the epigenetic regulation typically result in embryonic lethality or pathology (10), underscoring their essentiality for life. Moreover, the dynamic nature of epigenetic modifications places them at the interface of genetic, developmental and environmental interactions that ultimately engender phenotype.

This has spurred global initiatives to map epigenetic modifications across developmental- and disease-contexts, and correlate them with transcriptomes, genome architecture and genetic variation, among others (4, 11). For example, histone H3 lysine 4 trimethylation (H3K4me3) and lysine 27 acetylation (H3K27ac) are typically enriched specifically at gene promoters that become transcriptionally active in normal or disease cell types. Conversely, H3K9me2, H3K27me3, H2AK119ub and DNA methylation are often correlated with transcriptional repression, whilst H3K36me3 is enriched over transcribed gene bodies (12). Such pioneering studies have empowered unprecedented insight into genome function and revealed that chromatin modifications are important for controlling gene expression levels and normal cell function.

In support of the key role of chromatin modifications, manipulation of enzymes that catalyze H3K27me3 (histone 3 lysine 27 trimethylation), H2A119Kub, H3K4me3, H3K36me3, H3K9me3 and DNAme, all trigger widespread gene mis-expression, and embryonic lethality (1, 7-9). Moreover, changes in epigenetic landscapes are also directly linked with multiple diseases, including cancer and aging (3). Nevertheless, separating direct from indirect effects has proved challenging. Indeed, deciphering the quantitative impact of, and attributing causality to, chromatin marks per se has been confounded by pleiotropic effects of global dysregulation, as well as by non-histone substrates and non-catalytic functions of chromatin modifiers. For example, despite a tight association between H3K4me1/H3K27ac and active enhancers, recent evidence indicates that these marks may play a relatively minor role in enhancer function (10, 11). The field must therefore shift from mapping correlative changes in these marks to defining their causal and context-dependent function with high precision, which will be critical to understand disease mechanisms. Beyond that, there is a need to develop tools to specifically ‘edit’ chromatin modifications at specific loci, with a view to precisely reversing or manipulating abberant gene activity in disease that they cause.

The emergence of epigenetic editing technologies that permit site-specific modulation of chromatin modifications could effectively meet these challenges. One of the current tools fuses a chromatin-modifying protein to a nuclease-dead (d) Cas9, which targets a locus via a guide (g)RNA (12).

Nakamura M et al. (in: CRISPR technologies for precise epigenome editing. Nat Cell Biol. 2021 January; 23(1):11-22. doi: 10.1038/s41556-020-00620-7. Epub 2021 Jan. 8. PMID: 33420494) disclose that the epigenome involves a complex set of cellular processes governing genomic activity. Dissecting this complexity necessitates the development of tools capable of specifically manipulating these processes. The repurposing of prokaryotic CRISPR systems has allowed for the development of diverse technologies for epigenome engineering. They review the state of currently achievable epigenetic manipulations along with corresponding applications. They conclude that with future optimization, CRISPR-based epigenomic editing stands as a set of powerful tools for understanding and controlling biological function.

Goell J H, and Hilton I B (in: CRISPR/Cas-Based Epigenome Editing: Advances, Applications, and Clinical Utility. Trends Biotechnol. 2021 July; 39(7):678-691. doi: 10.1016/j.tibtech.2020.10.012. Epub 2021 May 7. PMID: 33972106) disclose that the epigenome dynamically regulates gene expression and guides cellular differentiation throughout the lifespan of eukaryotic organisms. Recent advances in clustered regularly interspaced palindromic repeats (CRISPR)/Cas-based epigenome editing technologies have enabled researchers to site-specifically program epigenetic modifications to endogenous DNA and histones and to manipulate the architecture of native chromatin. As a result, epigenome editing has helped to uncover the causal relationships between epigenetic marks and gene expression. As epigenome editing tools have continued to develop, researchers have applied them in new ways to explore the function of the epigenome in human health and disease. They discuss the recent technical improvements in CRISPR/Cas-based epigenome editing that have advanced clinical research and examine how these technologies could be improved for greater future utility.

Okada M, et al. (in: Stabilization of Foxp3 expression by CRISPR-dCas9-based epigenome editing in mouse primary T cells. Epigenetics Chromatin. 2017 May 8; 10:24. doi: 10.1186/s13072-017-0129-1. PMID: 28503202; PMCID: PMC5422987) disclose that epigenome editing is expected to manipulate transcription and cell fates and to elucidate the gene expression mechanisms in various cell types. For functional epigenome editing, assessing the chromatin context-dependent activity of artificial epigenetic modifier is required. They applied clustered regularly interspaced short palindromic repeats (CRISPR)-dCas9-based epigenome editing to mouse primary T cells, focusing on the Forkhead box P3 (Foxp3) gene locus, a master transcription factor of regulatory T cells (Tregs). The Foxp3 gene locus is regulated by combinatorial epigenetic modifications, which determine the Foxp3 expression. Foxp3 expression is unstable in transforming growth factor beta (TGF-β)-induced Tregs (iTregs), while stable in thymus-derived Tregs (tTregs). To stabilize Foxp3 expression in iTregs, the authors introduced dCas9-TET1CD (dCas9 fused to the catalytic domain (CD) of ten-eleven translocation dioxygenase 1 (TET1), methylcytosine dioxygenase) and dCas9-p300CD (dCas9 fused to the CD of p300, histone acetyltransferase) with guide RNAs (gRNAs) targeted to the Foxp3 gene locus. Although dCas9-TET1CD induced partial demethylation in enhancer region called conserved non-coding DNA sequences 2 (CNS2), robust Foxp3 stabilization was not observed. In contrast, dCas9-p300CD targeted to the promoter locus partly maintained Foxp3 transcription in cultured and primary T cells even under inflammatory conditions in vitro. Furthermore, dCas9-p300CD promoted expression of Treg signature genes and enhanced suppression activity in vitro. They conclude that artificial epigenome editing modified the epigenetic status and gene expression of the targeted loci, and engineered cellular functions in conjunction with endogenous epigenetic modification, suggesting effective usage of these technologies, which help elucidate the relationship between chromatin states and gene expression.

Huang Y H, et al. (in: DNA epigenome editing using CRISPR-Cas SunTag-directed DNMT3A. Genome Biol. 2017 Sep. 18; 18(1):176. doi: 10.1186/s13059-017-1306-z. PMID: 28923089; PMCID: PMC5604343) disclose that DNA methylation has widespread effects on gene expression during development. However, our ability to assign specific function to regions of DNA methylation is limited by the poor correlation between global patterns of DNA methylation and gene expression. They utilize nuclease-deactivated Cas9 protein fused to repetitive peptide epitopes (SunTag) recruiting multiple copies of antibody-fused de novo DNA methyltransferase 3A (DNMT3A) (dCas9-SunTag-DNMT3A) to amplify the local DNMT3A concentration to methylate genomic sites of interest. They demonstrate that dCas9-SunTag-DNMT3A dramatically increases CpG methylation at the HOXA5 locus in human embryonic kidney (HEK293T) cells. Furthermore, using a single guide RNA, dCas9-SunTag-DNMT3A is able to methylate a 4.5-kb genomic region and repress HOXA5 gene expression. Reduced representation bisulfite sequencing and RNA-seq show that dCas9-SunTag-DNMT3A methylates regions of interest with minimal impact on the global DNA methylome and transcriptome. They conclude that the effective and precise tools as discussed enable site-specific manipulation of DNA methylation and may be used to address the relationship between DNA methylation and gene expression.

CN111748583A discloses an inducible DNA methylation editing system based on CRISPR/dCas9, characterized in that: comprising a guide element, an anchoring element and an editing effect element which can act in sequence, the anchoring element comprises a stimulus responsive protein A and an inactivated SpCas 9, the editing effector elements comprise stimulus response protein B and DNA methylation editing effector protein, and the stimulus response protein A and the stimulus response protein B can be combined with each other under the stimulus effect, and the combination is released after the stimulus effect disappears.

WO 2018/053037A1 discloses compositions and methods for the delivery of enhanced demethylation activity to target DNA sequences in a mammalian cell. The compositions and methods are, useful for activity modulation of a targeted gene, or to create a gene regulatory network.

Vojta et al. (in: Repurposing the CRISPR-Cas9 system for targeted DNA methylation, Nucl Acid Res 2016 vol. 44, no. 12, doi:10.1093/nar/gkw159, ISSN 0305-1048, pages 5615-5628) developed a CRISPR-Cas9-based tool for specific DNA methylation consisting of deactivated Cas9 (dCas9) nuclease and catalytic domain of the DNA methyltransferase DNMT3A targeted by co-expression of a guide RNA to any 20 bp DNA sequence followed by the NGG trinucleotide. They demonstrated targeted CpG methylation in a ˜35 bp wide region by the fusion protein. They also showed that multiple guide RNAs could target the dCas9-DNMT3A construct to multiple adjacent sites, which enabled methylation of a larger part of a gene promoter. DNA methylation activity was specific for the targeted region and heritable across mitotic divisions. Finally, they demonstrated that directed DNA methylation of a wider promoter region of the target loci IL6ST and BACH2 decreased their expression.

A nuclease-defective or nuclease-deficient Cas9 protein (e.g., dCas9) with mutations on its nuclease domains retains DNA binding activity when complexed with sgRNA. dCas9 protein can tether and localize effector domains or protein tags by means of protein fusions to sites matched by sgRNA, thus constituting an RNA-guided DNA binding enzyme. dCas9 can be fused to transcriptional activation domain (e.g., VP64) or repressor domain (e.g., KRAB), and be guided by sgRNA to activate or repress target genes, respectively. dCas9 can also be fused with fluorescent proteins and achieve live-cell fluorescent labeling of chromosomal regions. Also, in cases where multiple copies of protein tags or effector fusions are necessary to achieve some biological threshold or signal detection threshold, multimerization of effector or protein tags by direct fusion with dCas9 protein is technically limited, by constraints such as difficulty in delivering the large DNA encoding such fusions, or difficulty in translating or translocating such large proteins into the nucleus due to protein size.

O'Geen H, et al. (in: dCas9-based epigenome editing suggests acquisition of histone methylation is not sufficient for target gene repression. Nucleic Acids Res. 2017 Sep. 29; 45(17):9901-9916. doi: 10.1093/nar/gkx578. PMID: 28973434; PMCID: PMC5622328) disclose that distinct epigenomic profiles of histone marks have been associated with gene expression, but questions regarding the causal relationship remain. They investigated the activity of a broad collection of genomically targeted epigenetic regulators that could write epigenetic marks associated with a repressed chromatin state (G9A, SUV39H1, Kruppel-associated box (KRAB), DNMT3A as well as the first targetable versions of Ezh2 and Friend of GATA-1 (FOG1)). dCas9 fusions produced target gene repression over a range of 0- to 10-fold that varied by locus and cell type. dCpf1 fusions were unable to repress gene expression. The most persistent gene repression required the action of several effector domains; however, KRAB-dCas9 did not contribute to persistence in contrast to previous reports. A ‘direct tethering’ strategy attaching the Ezh2 methyltransferase enzyme to dCas9, as well as a ‘recruitment’ strategy attaching the N-terminal 45 residues of FOG1 to dCas9 to recruit the endogenous nucleosome remodeling and deacetylase complex, were both successful in targeted deposition of H3K27me3. Surprisingly, however, repression was not correlated with deposition of either H3K9me3 or H3K27me3. Their results suggest that so-called repressive histone modifications are not sufficient for gene repression.

Chen et al. (in: Evelyn Chen, Enrique Lin-Shiao, Mohammad Saffari Doost, Jennifer A. Doudna Decorating chromatin for enhanced genome editing using CRISPR-Cas9 bioRxiv 2022.03.15.484540; doi: https://doi.org/10.1101/2022.03.15.484540) describe that CRISPR-associated (Cas) enzymes have revolutionized biology by enabling RNA-guided genome editing. Homology-directed repair (HDR) in the presence of donor templates is currently the most versatile way to introduce precise edits following CRISPR-Cas-induced double-stranded DNA cuts, but HDR efficiency is generally low relative to end-joining pathways that lead to insertions and deletions (indels). They tested the hypothesis that HDR could be increased using a Cas9 construct fused to PRDM9, a chromatin remodeling factor that deposits histone methylations H3K4me3 and H3K36me3 shown to mediate homologous recombination in human cells. The results show that the fusion protein contacts chromatin specifically at the Cas9 cut site in DNA to double the observed HDR efficiency and increase the HDR:indel ratio by 3-fold compared to that induced by Cas9 alone. HDR enhancement occurred in multiple cell lines with no increase in off-target genome editing. These findings underscore the importance of chromatin structure for the choice of DNA repair pathway during CRISPR-Cas genome editing and provide a new strategy to increase the efficiency of HDR.

Morita S, et al. (in: Targeted DNA demethylation in vivo using dCas9-peptide repeat and scFv-TET1 catalytic domain fusions. (2016) Nat Biotechnol 34: 1060-1065) disclose that despite the importance of DNA methylation in health and disease, technologies to readily manipulate methylation of specific sequences for functional analysis and therapeutic purposes are lacking. They adapt the previously described dCas9-SunTag for efficient, targeted demethylation of specific DNA loci. The original SunTag consists of ten copies of the GCN4 peptide separated by 5-amino-acid linkers. To achieve efficient recruitment of an anti-GCN4 scFv fused to the ten-eleven (TET) 1 hydroxylase, which induces demethylation, they changed the linker length to 22 amino acids. The system attains demethylation efficiencies >50% in seven out of nine loci tested. Four of these seven loci showed demethylation of >90%. They demonstrate targeted demethylation of CpGs in regulatory regions and demethylation-dependent 1.7- to 50-fold upregulation of associated genes both in cell culture (embryonic stem cells, cancer cell lines, primary neural precursor cells) and in vivo in mouse fetuses.

WO 2018/035495A1 relates to methods of modifying DNA methylation by contacting a genomic DNA sequence with a catalytically inactive site-specific nuclease fused to an effector domain having methylation or demethylation activity and one or more guide sequences.

To date, studies have applied epigenetic editing to a subset of specific loci and inferred causal biological insight, generally by measuring bulk expression changes (13). Such approaches to decode the epigenome have also gathered interest for biomedical and preclinical applications, such as reversing epigenome-dependent disease phenotypes (14). However, to enable the quantitative principles of chromatin function to be understood (or desirably manipulated), we must now (i) enhance the ON-target activity of these systems, (ii) expand capabilities to target multiple marks and combinations thereof, (iii) measure effects at single-cell resolution to capture the distribution of responses, and perhaps most critically, (iv) maximize throughput to more than hundreds of loci to dissect context-dependent responses systematically. Systematic and genome-wide association studies (GWAS) have proved powerful tools to advance genome research towards the goal of predicting genotype-to-phenotype relationships. Such efforts have implicated sequence variants (SNP, indel) located in promoters and cis regulatory elements (cREs) as the principal source of phenotype variation, implying human complex traits often manifest through genetic differences in gene regulation (rather than coding sequences) (15, 16). How diverse promoters/cRE and their cis-variants interact with epigenetic mechanisms to modulate gene activity, and ultimately phenotype, is thus a frontier challenge for understanding genome function, disease susceptibility, and evolutionary processes. Indeed, DNA sequence variation and epigenetic systems are intricately linked; chromatin states can impact sequence-dependent transcription factor (TF) occupancy whilst conversely DNA sequence influences chromatin states (17, 18). The pressing need to understand genome function and the molecular mechanisms that underlie a multitude of biological processes has been accelerated by genome-scale perturbation strategies, such as pooled CRISPR screening, by many groups including the inventors' (19-21). Advances to this have facilitated multi-parametric readouts from CRISPR-based screens (22-25). In particular, the targeted perturb-seq (TAP-seq) approach (26) enables expression of 1000s of target genes to be accurately quantitated in single-cells carrying a specific perturbation, with 1000s of perturbations across the population. Adapting such high-resolution approaches to uncover the regulatory logic by which chromatin-based systems intersect with DNA sequence, cis variants and cell identity to yield quantitative gene control, would be a key milestone towards unravelling genotype-to-phenotype interactions, as well as chromatin function.

Chromatin modifications are recognized as one of the key regulatory mechanisms for transcription control in normal and disease settings. Nonetheless, the precise causal function of specific chromatin modifications has proved challenging to dissect, i.e., the field has been limited in its technological capacity to interrogate the causal transactions of epigenetic modifications precisely, quantitatively, and within distinct genomic features. Even less is known about how chromatin states interface with diverse DNA sequences or cis variants to quantitatively impact transcription, and how cell environment affects this. Understanding the causal link between epigenetic marks and gene expression remains a central question in chromatin biology especially as recent advances in epigenome editing techniques are beginning to shed new light on these processes. The discovery of CRISPR-Cas9 interference, by targeting a catalytically dead mutant of Streptococcus pyogenes SpCas9 (dCas9) to block transcription, has provided a valuable tool for regulating gene expression. Several strategies have since fused dCas9 to well-characterized repressors and activators (e.g., KRAB and VP64) to modulate gene expression with enhanced silencing and activation capacity. Furthermore, novel tagging approaches have allowed more efficient recruitment of multiple effectors to a single-dCas9 anchor bound to a specific genomic locus. Recruitment strategies have also been combined with chemically inducible approaches to provide temporal control of transcriptional regulation. Finally, recent studies have also focused on regulatory DNA sequences, via the recruitment of dCas9 fused to the histone acetyl-transferase p300 or dCas9 fused to the DNA demethylase Tet1 to activate enhancers (Braun, S. M. G., Kirkland, J. G., Chory, E. J. et al. Rapid and reversible epigenome editing by endogenous chromatin regulators. Nat Commun 8, 560 (2017). https://doi.org/10.1038/s41467-017-00644-y). There is thus a clear need for a large-scale, targeted perturbation strategy to dissect the causal regulatory function of chromatin marks across endogenous contexts, in order to ultimately apply the technology to clinical scenarios and therapy.

It is an object of the present invention to provide an additional tool stemming from the above for the field of epigenetics, methylation, precision genome control, and even the therapy of related diseases. Other objects and advantages will become apparent upon further studying the present specification with reference to the accompanying examples.

In a first aspect thereof, the object of the present invention is solved by providing an, in particular proteinaceous, complex comprising i) a catalytically inactive site-specific nuclease linked to ii) an array of between two and ten, preferably three to seven effector domains each having a specific chromatin modifying activity, such as, for example, a specific DNA methylation activity, a histone methylation activity, a specific histone acetylation or ubiquitination activity, and/or a specific chromatin demethylation/deacetylation activity, wherein the effector domains are each separated by a linker providing sufficient distance between the domains and the nuclease in order not to substantially interfere with their specific chromatin modifying activities, and the binding of the site-specific nuclease.

Preferred is the complex according to the present invention, wherein the proteinaceous complex comprises a fusion protein of the nuclease linked to a protein sequence comprising three to seven effector domain binding motifs that are each separated by a linker sequence, for example, Streptococcus pyogenes dCas9^{GCN4}₍₃₋₇₎. The complex may further comprise a number of effector domains, each non-covalently bound to a binding motif, and the complex optionally further comprising at least one suitable guide RNA (gRNA).

Further preferred is the complex according to the present invention, wherein the chromatin modifying activity is histone methylation, such as, for example histone methylation contributing to stable or reversible gene expression control.

In a second aspect thereof, the object of the present invention is solved by providing a set of nucleic acids, each encoding for at least one of the proteins and/or the guide RNA (gRNA) and/or the tagBFP of the complex according to the present invention.

In a third aspect thereof, the object of the present invention is solved by providing a set of genetic constructs, such as expression vectors, comprising the set of nucleic acids according to the present invention, wherein preferably each nucleic acid encoding for an effector domain, CD and/or CD^{scFV} comprises an inducible promotor, such as a tet-responsive promoter. More preferably, the constructs according to the present invention are viral constructs, such as viral constructs, for example derived from adeno associated virus (AAV), lentiviruses or retroviruses.

In a fourth aspect thereof, the object of the present invention is solved by providing a recombinant cell, comprising the set of nucleic acids according to the present invention, and/or the set of genetic constructs according to the present invention.

In a fifth aspect thereof, the object of the present invention is solved by providing a method for producing the complex according to the present invention, comprising expressing the set of nucleic acids according to the present invention, and/or the set of genetic constructs according to the present invention in the recombinant cell according to the present invention, optionally comprising the step of inducing expression, for example using a tetracycline.

In a sixth aspect thereof, the object of the present invention is solved by providing a method for specifically epigenetically modifying chromatin in a cell, tissue, cellular nucleus, and/or sample comprising chromatin, comprising introducing into the cell, tissue, cellular nucleus, and/or sample the complex according to the present invention, and one or more guide RNA, thereby specifically epigenetically modifying chromatin in the cell, tissue, cellular nucleus, and/or sample. Preferably, the epigenetic modification comprises histone methylation, DNA methylation, histone acetylation, histone ubiquitination, DNA demethylation, histone deacetylation, multiplexed epigenetic editing of histones, H3K9me2/3+DNA methylation, H3K4me3+H3K36me3, H3K4me3+H3K79me2, H3K36me3+H3K79me2, H3K9me2/3+H4K20me3, bivalent epigenetic editing of histones, and/or polycomb epigenetic editing of histones.

In a seventh aspect thereof, the object of the present invention is solved by providing a method for modulating the expression of at least one target DNA sequence in a cell, tissue, cellular nucleus, and/or sample comprising chromatin, said method comprising: introducing into the cell, tissue, cellular nucleus, and/or sample the complex according to the present invention, and one or more guide RNA sequence that is specific for the at least one target DNA sequence, thereby specifically epigenetically modulating the expression of at least one target DNA sequence in the cell, tissue, cellular nucleus, and/or sample. Preferably, the at least one target DNA sequence comprises a nucleic acid sequence that is specific for a condition and/or disease state related to epigenetically modified chromatin, such as, for example, genetic disorders, proliferative disorders, such as cancer, immune cells that produce autoantibodies, bacterial or viral infections, protozoan infections, fragile-X syndrome, muscular dystrophy, kidney injury, cardiovascular diseases, shortened organismal lifespan, tissue aging, neurodegenerative disorders, such as Alzheimer's disease (AD), Parkinson's disease (PD), Huntington's disease (HD), Amyotrophic lateral sclerosis (ALS), drugs of abuse, including alcohol abuse disorder, epigenetic diseases, including imprinting disorders, such as Prader-Willi Syndrome.

In an eighth aspect thereof, the object of the present invention is solved by providing a method for detecting the biological effect of specifically epigenetically modifying chromatin in a cell, tissue, cellular nucleus, and/or sample comprising chromatin and/or modulating the expression of at least one target DNA sequence in a cell, tissue, cellular nucleus, and/or sample comprising chromatin, comprising performing the method according to the present invention, and detecting at least one biological effect in the cell, tissue, cellular nucleus, and/or sample comprising chromatin, wherein said biological effect is selected from the group consisting of changes in gene expression, changes in the amount of a protein, cis-genetic effects, changes in nucleic acid splicing, changes in the nuclear positioning of loci, changes in the formation and disruption of TADs, changes in the termination site, activating a promotor, repressing a promotor, changes in genetic-epigenetic interactions, functional relation between genetic variants and the epigenetic state of chromatin, changes in inherited methylation and imprinting, and linking a specific epigenetic change with a disease or cellular phenotype.

In a ninth aspect thereof, the object of the present invention is solved by providing a cell having a specifically epigenetically modified chromatin, produced by performing the method according to the present invention, and, optionally, isolating said cell, wherein preferably the cell is a stem cell, a neuron, a post-mitotic cell, or a fibroblast, and/or wherein the cell is an animal cell, such as mammalian cell, preferably human or rodent cell.

In a tenth aspect thereof, the object of the present invention is solved by providing a method for identifying an agent specifically epigenetically modifying chromatin in a cell, tissue, cellular nucleus, and/or sample comprising chromatin, an agent modulating the expression of at least one target DNA sequence in a cell, tissue, cellular nucleus, and/or sample comprising chromatin and/or biological effect of specifically epigenetically modifying chromatin in a cell, tissue, cellular nucleus, and/or sample comprising chromatin, comprising performing the method according to the present invention in the presence and absence of a test agent, wherein the test agent is identified as an agent specifically epigenetically modifying chromatin in a cell, tissue, cellular nucleus, and/or sample comprising chromatin, an agent modulating the expression of at least one target DNA sequence in a cell, tissue, cellular nucleus, and/or sample comprising chromatin and/or biological effect of specifically epigenetically modifying chromatin in a cell, tissue, cellular nucleus, and/or sample comprising chromatin, if the modulation and/or biological effect in the presence of the agent differs from the modulation and/or biological effect in the absence of the agent or to a control.

In an eleventh aspect thereof, the object of the present invention is solved by providing a method for preventing or treating a disease related to epigenetically modified chromatin, such as, for example, genetic disorders, proliferative disorders, such as cancer, immune cells that produce autoantibodies, bacterial or viral infections, protozoan infections, fragile-X syndrome, muscular dystrophy, kidney injury, cardiovascular diseases, shortened organismal lifespan, tissue aging, neurodegenerative disorders, such as Alzheimer's disease (AD), Parkinson's disease (PD), Huntington's disease (HD), Amyotrophic lateral sclerosis (ALS), drugs of abuse, including alcohol abuse disorder, epigenetic diseases, including imprinting disorders, such as Prader-Willi Syndrome in a subject in need of such prevention or treatment, comprising administering to a subject in need of such treatment an effective amount of at least one of the complex according to the present invention, and one or more suitable guide RNA sequences, the set of nucleic acids according to the present invention, the set of genetic constructs, such as expression vectors, according to the present invention, the cell according to the present invention, and/or the agent as identified according to the present invention.

Another preferred aspect relates to the at least one of the complex according to the present invention, and one or more suitable guide RNA sequences, the set of nucleic acids according to the present invention, the set of genetic constructs, such as expression vectors, according to the present invention, the cell according to the present invention, and or the agent as identified according to the present invention for use in the prevention and/or treatment of diseases, or for use in the prevention and/or treatment of diseases related to epigenetically modified chromatin, such as, for example, genetic disorders, proliferative disorders, such as cancer, immune cells that produce autoantibodies, bacterial or viral infections, protozoan infections, fragile-X syndrome, muscular dystrophy, kidney injury, cardiovascular diseases, shortened organismal lifespan, tissue aging, neurodegenerative disorders, such as Alzheimer's disease (AD), Parkinson's disease (PD), Huntington's disease (HD), Amyotrophic lateral sclerosis (ALS), drugs of abuse, including alcohol abuse disorder, epigenetic diseases, including imprinting disorders, such as Prader-Willi Syndrome in a subject in need of such prevention or treatment.

In a twelfth aspect thereof, the object of the present invention is solved by providing the Use of the complex according to the present invention, the set of nucleic acids according to the present invention, the set of genetic constructs, such as expression vectors, according to the present invention, and/or the cell according to the present invention, for specifically epigenetically modifying chromatin in a cell, tissue, cellular nucleus, and/or sample comprising chromatin according to the present invention, for modulating the expression of at least one target DNA sequence in a cell, tissue, cellular nucleus, and/or sample comprising chromatin according to the present invention, detecting the biological effect of specifically epigenetically modifying chromatin in a cell, tissue, cellular nucleus, and/or sample according to the present invention, and/or identifying an agent according to the present invention.

As mentioned above, in a first aspect thereof, the object of the present invention is solved by an, in particular proteinaceous, complex comprising
i) a catalytically inactive site-specific nuclease linked to
ii) an array of between two and ten, preferably three to seven effector domains each having a specific chromatin modifying activity,
wherein the effector domains are each separated by a linker providing sufficient distance between the domains and the nuclease in order not to substantially interfere with their specific chromatin modifying activities, and the binding of the site-specific nuclease.

The specific chromatin modifying activity is preferably selected from a specific DNA methylation activity, a histone methylation activity, a specific histone acetylation or ubiquitination activity, and/or a specific chromatin demethylation/deacetylation activity

The present invention provides an improved epigenome editing platform, to systematically program specific- and combinatorial-chromatin modifications across tens-of-thousands of contexts in living cells. This resource can be used to capture multi-modal functional responses at allelic, single-cell resolution, from diverse lineages. The unprecedented scale of precision perturbations will help to uncover the regulatory logic by which distinct chromatin modifications interact with genomic features, sequence variants, and cellular identity, to shape quantitative gene expression patterns, and to further identify the trans-acting and cis-structural mechanisms that implement the functionality of epigenetics, and in particular chromatin marks. Moreover, the present invention can be deployed to reverse or manipulate abberant genome or chromatin states in disease.

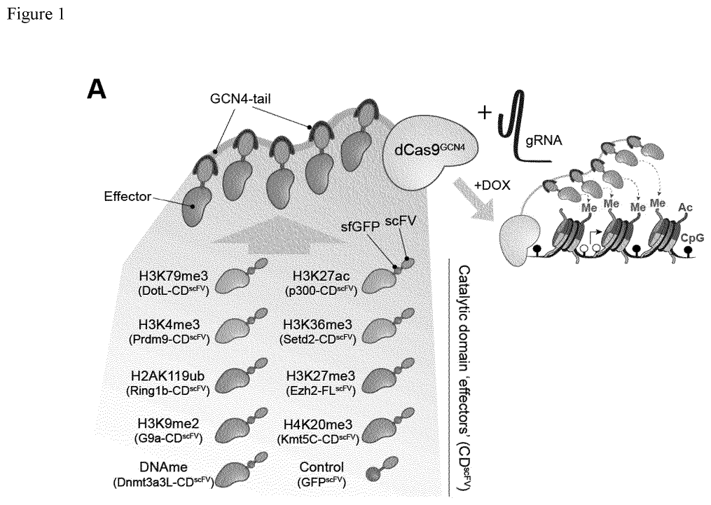

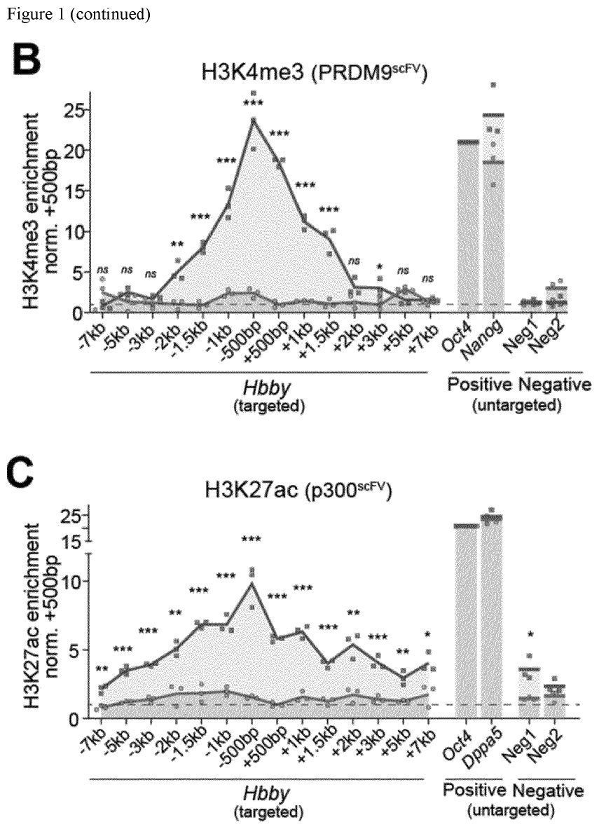

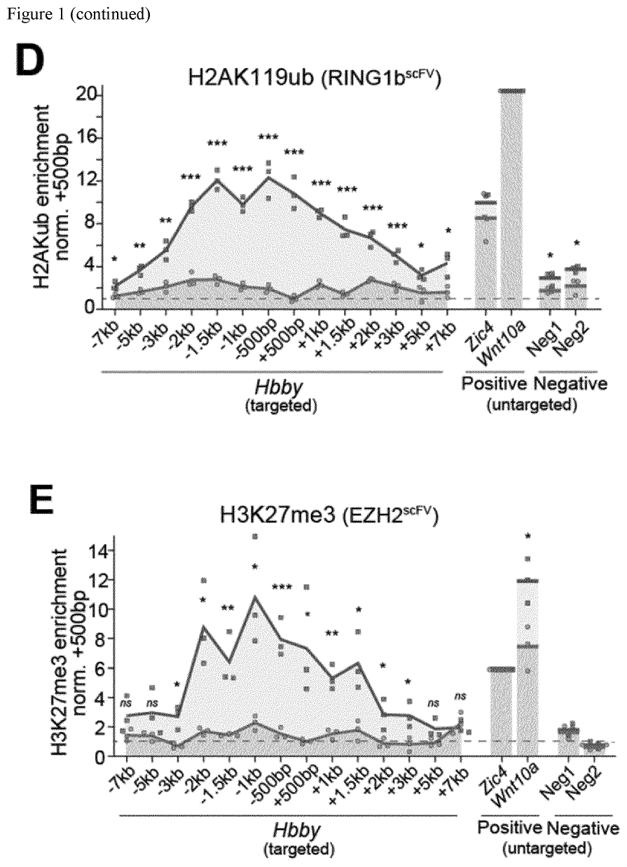

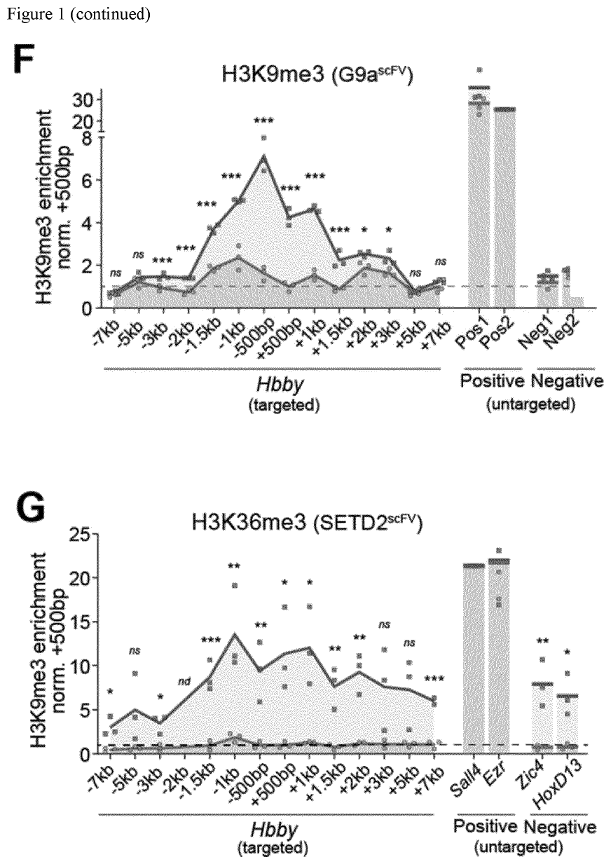

Developed was a modular CRISPR-based toolkit that in its current form can precisely and inducible program nine distinct epigenetic modifications to endogenous target loci. The ability to site-specifically deposit epigenetic markings, such as methylation including H3K27me3, H3K4me3, H3K79me3, H2AK119ub, and H3K36me3, represents a powerful gain-of-function perturbation strategy to explicitly assess their causal impact. In a preferred embodiment, the present tool provides dCas9 linked with an array of five GCN4 motifs (dCas9^{GCN4}), each separated by a linker sequence designed with optimal spacing to accommodate bulky proteins without sterically hindering their catalytic activity. This dCas9^{GCN4} can carry a number (e.g. five) ‘effector’ proteins or domains to a specific locus, wherein the effector domains are complexed via a GCN4-specific scFV domain (see FIG. 1A). The inventors have engineered and tested a comprehensive suite of effector domains that preferably comprise only the catalytic domain (collectively: CD^{scFV}) of chromatin-modifying enzymes, for example Setd2-CD^{scFV} for H3K36me3 and Prdm9-CD^{scFV} for H3K4me3.

The inventive complex has multiple other advantages built in that collectively result in an epigenetic editing platform technology with exceptional features to enable discovery.

The advantages include:

Highly active editing. The recruitment of e.g. five copies of a specific CD^{scFV} to a target locus greatly amplifies ON-target programming of chromatin modifications, both in amplitude and in genomic breadth. This ensures de novo histone methylation deposition that is comparable to strong endogenous peaks, facilitating both negative- and positive-functional effects.

Catalytic domain specificity. When using tailored effector domains, e.g. only the catalytic cores of these, the complexes avoid undesired side-effects of targeting full-length chromatin-modifying proteins. Since full-length proteins can have major non-catalytic functions and/or recruit other protein complexes, the preferred use of catalytic domains enables the function of targeted chromatin marks per se to be assessed (see also below).

Combinatorial epigenetic editing. The inventive complex and system is modular, and therefore (preferably up to five) different CD^{scFV} can be recruited simultaneously. This enables multiplexed and even tunable epigenetic editing that permits establishment of de novo domains of different chromatin modifications (e.g. bivalent or polycomb).

Minimized off-targeting. Because the effector domains and CD^{scFV} as used here are not directly fused to dCas9 and the CDs generally lack their endogenous DNA-binding domain, they exhibit minimal off-target activity.

Temporally-resolved. In the genetic constructs as provided in the context of the present invention, each effector domain and/or CD^{scFV} is dynamically induced via a DOX-responsive promoter and may also carry a protein destabilization (d2) domain, facilitating rapid degradation upon DOX-withdrawal. This enables interrogation of temporal responses and epigenetic memory (persistence of chromatin).

Dynamic tracking. Preferably, some or all effector groups are linked with superfolder GFP (sfGFP), and some or all gRNAs with tagBFP, allowing this system to be tracked in real-time, for cells to be purified, and testing of drug dose-dependent responses (e.g. comparing GFP^{low} and GFP^{high} cells).

Controls for indirect/confounding effects. As further controls, effectors domains identical to the CD^{scFV} one but containing single-point mutations which abrogate their enzymatic activity were also generated (catalytic mutant effectors, mut-CD^{scFV}). These include: Dot11-mut-CD^{scFV}, p300-mut-CD^{scFV}, Prdm9-mut-CD^{scFV}, Setd2-mut-CD^{scFV}, Ring1b-mut-CD^{scFV}, Ezh2-mut-CD^{scFV}, G9a-mut-CD^{scFV}, Kmt5C-mut-CD^{scFV}, Dnmt3a3L-mut-CD^{scFV}. The mutant effectors are unable to write chromatin modifications on histones and DNA. Therefore, they represent important controls to make sure that, for example, a change in gene expression is caused exclusively by the deposition of chromatin modification(s) per se rather than by some indirect event such as the recruitment of other protein complexes (change in gene expression is achieved only when the wild-type CD^{scFV}, but not the mutant mut-CD^{scFV}, is recruited). Additionally, a GFP-only effector (GFP^{scFV}) has also been made to control for steric hindrance effects caused by the recruitment on chromatin of the bulky dCas9^{GCN4} protein.

Taken together, these features permit carefully controlled assessment of the functional implications of introducing specific chromatin modification(s) per se at any given locus, whilst ruling out confounding effects. They also permit the capacity to introduce or reverse specific chromatin modifications in living cells, towards reversing pathological outcomes.

Policarpi C, Dabin J, and Hackett J A (in: Epigenetic editing: Dissecting chromatin function in context. Bioessays. 2021 May; 43(5):e2000316. doi: 10.1002/bies.202000316. Epub 2021 Mar. 16. PMID: 33724509), amongst others, review the SpdCas9-SunTag technology consisting of a SpdCas9 enzyme fused to several (5-20) tandem repeats of the GCN4 binding motif (SunTag), which recruits any proteins coupled with a small-chain variable fragment (scFV) domain. This system allows recruitment of multiple copies of the same effector domain (or of different ones) to a specific genomic site, thus promoting high ON-target editing efficiency and increased spreading of the induced epigenetic state, while minimizing OFF-target effects. The same strategy could be used in the future to dissect how combinatorial chromatin modifications influence transcription and genome regulation. Nevertheless, the dCas9-SunTag system as described is a fusion between the dCas9 protein to a tail of GCN4 peptides that can recruit up to 10 copies of scFV-VP64, which amplifies the activation signal (Tanenbaum M. E., Gilbert L. A., Qi L. S., Weissman J. S., Vale R. D. A protein-tagging system for signal amplification in gene expression and fluorescence imaging. Cell. 2014; 159:635-646). Also, a spacer as short as five amino acids is disclosed as sufficiently separating peptides to allow binding of antibodies to neighboring peptides.

WO 2021/247570 discloses fusion proteins, compositions and methods for manipulation of genomes of living organisms. The fusion comprises, from N-terminus to C-terminus, a DNA methyltransferase domain, a first XTEN linker, a nuclease-deficient RNA-guided endonuclease enzyme, a second XTEN linker, and a Kruppel-associated box domain.

WO 2014/197748 discloses Clustered Regularly Interspaced Short Palindromic Repeats (CRISPR)/CRISPR-associated (Cas) 9-based system related compositions and methods of using said CRISPR/Cas9-based system related compositions for altering gene expression and genome engineering. Also disclosed are compositions and methods of using said compositions for altering gene expression and genome engineering in muscle, such as skeletal muscle and cardiac muscle. These tools use a single fusion protein comprising an essential KRAB (Kruppel-associated box domain). The present system does not require KRAB, and is more specific (less “off-target effects” with more “fine-tuning” becoming possible).

Carlini V, Policarpi C, and Hackett J A (in: Epigenetic inheritance is gated by naïve pluripotency and Dppa2. EMBO J. 2022 Apr. 4; 41 (7):e108677. doi: 10.15252/embj.2021108677. Epub 2022 Feb. 24. PMID: 35199868; PMCID: PMC8982627) developed a CRISPR-based epigenetic programming tool. They employed a catalytically dead (d) Cas9 fused with an array of five optimally spaced GCN4 repeats (dCas9^{GCN4}). These serve as docking sites to recruit up to five “effectors” to a specific genomic locus via their single-chain antibody (scFv) domain. The modular system amplified both the quantitative level and domain size of ON-target epigenome editing, relative to dCas9 effector fusions, while minimizing OFF-target effects. To target de novo heterochromatin, they generated KRAB^{GFP-scFv} and DNMT3A/3L^{GFP-scFv} effectors, which promote direct deposition of H3K9me3 and DNA methylation, respectively. The present system has been drastically improved, and now allows for many different kinds of chromatin editing (see further below).

In the context of the present invention, a “proteinaceous complex” shall mean a complex of biological molecules that—at least to a substantial amount thereof—comprise peptide chains made of amino acids, such as proteins. The complex is formed by covalent and/or non-covalent bonds, and preferably includes molecules that are complexed by antigen/antigen-binding (e.g. antibody recognition-based) activities. The complex may furthermore contain other proteins, such as fluorescent labels, but also nucleic acids, like gRNA, and/or other chemical groups, such as linkers or coupling agents.

In yet another aspect of the methods and compounds according to the present invention, multiple labels and/or markers are used. Markers can be used both for the nucleic acid molecules that form part of the assays, as well as the protein components (e.g., nuclease and/or fusions). Labels and markers can be included into the components of the assays (in particular the nucleic acids and/or the proteins), as well as constitute moieties that are attached, either covalently or non-covalently.

In principle, any suitable catalytically inactive site-specific nuclease can be used in the context of the proteinaceous complex according to the present invention. As mentioned above, a nuclease-defective or nuclease-deficient Cas9 protein (e.g., dCas9) with mutations on its nuclease domains retains DNA binding activity when complexed with sgRNA. dCas9 protein can tether and localize effector domains or protein tags by means of protein fusions to sites matched by sgRNA, thus constituting an RNA-guided DNA binding enzyme. dCas9 can be fused to transcriptional activation domain (e.g., VP64) or repressor domain (e.g., KRAB), and be guided by sgRNA to activate or repress target genes, respectively. dCas9 can also be fused with fluorescent proteins and achieve live-cell fluorescent labeling of chromosomal regions. However, in such systems, only one Cas9-effector fusion is possible because sgRNA:Cas9 pairing is exclusive. Also, in cases where multiple copies of protein tags or effector fusions are necessary to achieve some biological threshold or signal detection threshold, multimerization of effector or protein tags by direct fusion with dCas9 protein is technically limited, by constraints such as difficulty in delivering the large DNA encoding such fusions, or difficulty in translating or translocating such large proteins into the nucleus due to protein size. Preferred is a catalytically inactive site-specific nuclease that is selected from the group consisting of a catalytically dead (d) Cas9 from Streptococcus pyogenes, asCas12, saCas9, miniCas9, dCas9, fCas9, SceI, and dCas9/fCas9 fusions. Since some preferred delivery systems, such as AAV only have a limited load capacity (i.e., can only accommodate a certain number and size of protein molecules) this has stimulated the development of smaller CRISPR/Cas9 systems, such as those built around Staphylococcus aureus saCas9, which is a preferred nuclease.

Preferred is the complex according to the present invention, wherein the complex comprises a fusion protein of the nuclease fused or linked to a protein sequence comprising three to seven effector domain binding motifs that are each separated by a second linker sequence, for example, Streptococcus pyogenes dCas9GCN4₍₃₋₇₎, and the complex optionally further comprising a number of effector domains, each bound to a binding motif, and the complex optionally further comprising at least one suitable guide RNA (gRNA).

An “effector domain” in the context of the present invention is a polypeptide or protein having at least one specific chromatin modifying activity. Preferred is an effector domain having a specific chromatin modifying activity, such as, for example, a specific DNA methylation activity, a histone methylation activity, a specific histone acetylation or ubiquitination activity, and/or a specific chromatin demethylation/deacetylation activity. The present invention also includes effector domains that exhibit their chromatin modifying activity in combination, so-called “combinatorial effectors”. The present invention advantageously also uses effectors that have been trimmed, i.e., comprising the catalytic domains (CD) of full-length chromatin modifying polypeptides or proteins, and/or more preferably the catalytic domains (CD) thereof fused to an effector domain binding motif-specific scFV domain (CDscFV). Again, this has further advantages in view of limited load delivery vehicles, like AAV-based vehicles, and was also found to increase the activity and specificity of the system. Other ways in which to deliver the system comprise non-viral delivery modes including: physical methods (e.g., electroporation, microinjection, sonoporation, and hydrodynamic delivery and chemical approaches (e.g., lipid particles, polymer nanoparticles, gold nanoparticles, and cell-penetrating peptides (CPPs)).

In the context of the present invention, the term effector domains shall also include polypeptides having at least 50%, preferably at least 70, more preferably at least 80%, more preferably at least 90%, and more preferably at least 95% identity with a polypeptide sequence as disclosed herein, and having the chromatin modifying activity as disclosed herein.

Further preferred in the context of the present invention is a complex according to the present invention, wherein the effector domain comprises a chromatin modifying protein or polypeptide selected from the group consisting of Dot1L (H3K79me2), p300 (H3K27ac), Prdm9 (H3K4me3), Setd2 (H3K36me3), Ring1b (H2AK119ub), Ezh2 (H3K27me3), G9a (H3K9me2), Kmt5C (H4K20me3), Dnmt3a3L (DNAme), OGT (GlcNAC), where possible the catalytic domains (CD) thereof, and where possible the catalytic domains (CD) thereof fused to an effector domain binding motif-specific scFV domain (CDscFV).

Histones can be modified in several ways (see also above), including acetylation, methylation (lysines), methylation (arginines), phosphorylation, ubiquitylation, sumoylation, ADP ribosylation, deimination, and proline isomerization. Preferred is the complex according to the present invention, wherein the chromatin modifying activity is histone methylation, such as, for example histone methylation contributing to stable or reversible gene expression control.

In the context of the present invention, preferred effector domains for histone modifications are selected from the group consisting of HAT1, CBP/P300, PCAF/GCN5, TIP60, HB01 (ScESA1, SpMST1), ScSAS3, ScSAS2 (SpMST2), ScRTT109, SirT2 (ScSir2), SUV39H1, SUV39H2, G9a, ESET/SETDB1, EuHMTase/GLP, CLL8, SpClr4, MLL1, MLL2, MLL3, MLL4, MLL5, SET1A, SET1B, ASH1, Sc/Sp SET1, SET2 (Sc/Sp SET2), NSD1, SYMD2, DOT1, Sc/Sp DOT1, Pr-SET 7/8, SUV4 20H1, SUV420H2, SpSet 9, EZH2, RIZ1, LSD1/BHC110, JHDMla, JHDM1b, JHDM2a, JHDM2b, JMJD2A/JHDM3A, JMJD2B, JMJD2C/GASC1, JMJD2D, CARM1, PRMT4, PRMT5, Haspin, MSK1, MSK2, CKII, Mst1, Bmi/Ring1A, RNF20/RNF40, and ScFPR4.

Preferably, the effector domain may be suitably labelled or tagged, for example with a fluorescent marker, such as sfGFP (super-folder GFP). sfGFP surprisingly helps recombinant/artificial proteins (such as according to the invention) to fold better and thus improves the inventive complex.

More preferably, the proteinaceous complex according to the present invention comprises three to seven effector domains, in particular five effector domains. The domains are at least two or more, at least three or more, at least four or more, or at least five or more different effector domains.

Further preferred is the proteinaceous complex according to the present invention, wherein the effector domain binding motif consists of an epitope comprising a peptide sequence of between 17 to 29 amino acids, preferably between 17 to 21 amino acids, and having little or no structural folding under physiological conditions. In principle, any suitable binding motif can be used, preferred is the GCN4 epitope motif sequence, because of specificity and stability. The GCN4 epitope motif sequence (or “GCN4 peptide”) comprises 22 amino acids (LLPKNYHLENEVARLKKLVGER; SEQ ID NO: 1). Nevertheless, the array of the effector domains (the “tail”) may also be complexed or fused to the nuclease in different ways, e.g., with chemical bonds.

In the complex according to the present invention, furthermore a linker is provided providing sufficient distance between the domains and between the domains and the nuclease in order to not substantially interfere with their specific chromatin modifying activities, and the binding of the site-specific nuclease, respectively. Linkers are known to the person of skill in the art, preferred is a length of the linker sequence that is between 25 and 19 amino acids, preferably 22, further preferably comprising glycine (G) and serine (S) amino acids. It was found that the linker in the dCas9-SunTag (5 amino acids) is insufficient for an optimal catalytic activity of enzymes. As long as the linker does not interfere with the desired activities, the sequence of the linker is not particularly important, the length/distance was found to be more important. Substantially in the context of the interference as before shall mean that the complex according to the present invention both exhibits a sufficient specific chromatin modifying activities and a sufficient binding of the site-specific nuclease.

More preferred is the complex according to the present invention, wherein the effector domains are bound via an effector domain binding motif-specific scFV domain, in particular a GCN4-specific scFV domain, wherein the scFV is optionally linked to the effector domain via an effector linker group, such as, for example a group comprising a fluorescent protein, such as a GFP or sfGFP protein, and wherein the effector domain may further be fused to a protein destabilization domain, such as, for example, d2.

Greer, Eric L, and Yang Shi (in: “Histone methylation: a dynamic mark in health, disease and inheritance.” Nature reviews. Genetics vol. 13, 5 343-57. 3 Apr. 2012, doi:10.1038/nrg3173) disclose that organisms require an appropriate balance of stability and reversibility in gene expression programs, to maintain cell identity or to enable responses to stimuli; epigenetic regulation is integral to this dynamic control. Post-translational modification of histones by methylation is an important and widespread type of chromatin modification that is known to influence biological processes in the context of development and cellular responses. They provide a broad overview of how histone methylation is regulated and leads to biological outcomes, to evaluate how histone methylation contributes to stable or reversible control. The importance of maintaining or reprogramming histone methylation appropriately is illustrated by links to disease and aging, or possibly transmission of traits across generations. Histone methylation occurs on all basic residues: arginines3, lysines4 and histidines5. Lysines can be mono (me1)4, di(me2)6, or tri(me3)7 methylated on their F amine group, arginines can be mono(me1)3, symmetrically dimethylated (me2s), or asymemetrically dimethylated (me2a) on their guanidinyl group8, and histidines have been reported to be monomethylated8,9 although this methylation appears to be rare and has not been further characterized. The most extensively studied histone methylation sites include histone H3 lysine 4 (H3K4), H3K9, H3K27, H3K36, H3K79 and H4K20. Sites of arginine methylation include H3R2, H3R8, H3R17, H3R26 and H4R3. However, many other basic residues throughout the histone proteins H1, H2A, H2B, H3 and H4 have also been recently identified as methylated.

A major advantage of the complex according to the present invention is the possibility to specifically “tune” the chromatin modifying activity based on a specific selection and thus combination of the effector domains as used, such as, for example, a specific DNA methylation activity, a histone methylation activity, a specific histone acetylation or ubiquitination activity, and/or a specific chromatin demethylation/deacetylation activity or combinations thereof. Thus, the complex may comprise at least two or more, at least three or more, at least four or more, or at least five or more different effector domains, such as, for example a combination of Setd2-CD^{scFV} for H3K36me3 and Prdm9-CD^{scFV} for H3K4me3, and preferably no KRAB and/or VPR effector domain. Further preferred is at least one chromatin modifying protein or polypeptide selected from the group consisting of Dot1L (H3K79me2), p300 (H3K27ac), Prdm9 (H3K4me3), Kmt2b (H3K4me3), Set1a (H3K4me3), Setd2 (H3K36me3), Ring1b (H2AK119ub), Ezh2 (H3K27me3), G9a (H3K9me2), Setdb1 (H3K9me3), Suv39h1 (H3K9me3), Kmt5C (H4K20me3), Dnmt3a3L (DNAme), Ogt (GlcNAC), Prmt5 (H4R3me2s), Hdac1/2/3/4 (histone deacetylases), Sirt1/2/3/6 (histone deacetylases), Kat2a (lysine acetyltransferase), Lsd1 (H3K4me demethylase), Kdm5a/b/c (H3K4 demethylase), Kdm2b (H3K4 and H3K79 demethylase), Tet1/2/3 (methylcytosine dioxygenases), Utx (H3K27 demethylase), JMJD3 (H3K27 demethylase), Kdm4a/b/c/d (H3K36 and H3K9 demethylase) the catalytic domains (CD) thereof, the catalytic domains (CD) thereof fused to an effector domain binding motif-specific scFV domain (CDscFV) or a fragment antigen-binding (Fab) domain.

Another aspect of the present invention then relates to a set of nucleic acids, each encoding for at least one of the proteins and/or the guide RNA (gRNA) and/or the tagBFP of the complex according to the present invention.

In the context of the present invention, the term nucleic acids shall also include sequences having at least 50%, preferably at least 70, more preferably at least 80%, more preferably at least 90%, and more preferably at least 95% identity with a sequence as disclosed herein, and also encoding for a peptide or polypeptide having substantially the same activity as the polypeptides as disclosed herein. For example, preferred is the complex according to the present invention wherein said guide RNA molecule comprises a sequence that is to at least 80%, preferably to more than 90%, more preferably to more than 95%, and most preferably 100% complementary to a target RNA.

Certain portions of the nucleic acid molecules as used in the methods according to the present invention are found and/or designed to specifically hybridize with complementary portions in other molecules. As known to the person of skill, for this, the hybridization and washing conditions are critical. If the sequences are 100% complementary, then a high stringency hybridization may be carried out. Nevertheless, according to the invention, the portions that hybridize and/or specifically hybridize are complementary to at least 80%, preferably complementary to more than 90%, more preferably to more than 95%, and most preferably are 100% complementary. The stringency of hybridization is determined by the hybridization temperature and the salt concentration in the hybridization buffer, and high temperature and low salt is more stringent. A commonly used washing solution is SSC (Saline Sodium Citrate, a mixture of NaCitrate and NaCl). Hybridization may be carried out in solution or—more commonly—at least one component may be on a solid-phase support, e.g., nitrocellulose paper. Frequently used protocols employ a blocking reagent, such as casein from nonfat dried milk or bovine serum albumin, often in combination with denatured, fragmented salmon sperm DNA (or any other heterologous DNA of high complexity) and a detergent, such as SDS. Often a very high concentration of SDS is used as a blocking agent. Temperatures may be between 42 and 65° C. or higher, and buffers may be 3×SSC, 25 mM HEPES, pH 7.0, 0.25% SDS final.

The set according to the present invention therefore encodes at least two, preferably three, four, five or all of the proteinaceous components of the complex according to the present invention. Preferred examples are a first set comprising nucleic acids encoding the catalytically inactive site-specific nuclease comprising the linker structure (“tail”), which may be genetically fused to the nuclease enzyme, and/or a second set comprising nucleic acids encoding the effector domain polypeptides as used. Further preferred sets are sets comprising nucleic acids encoding between two and ten, preferably three to seven effector domains each having a specific chromatin modifying activity, such as, for example, a specific DNA methylation activity, a histone methylation activity, a specific histone acetylation or ubiquitination activity, and/or a specific chromatin demethylation/deacetylation activity. Other preferred sets include nucleic acids encoding at least one suitable guide RNA (gRNA). The sets may be combined further with a nucleic acid encoding a tagBFP for co-expression and then tagging the gRNA.

Preferred sets further include nucleic acids encoding effector domain binding motif-specific scFV domains, in particular the GCN4-specific scFV domain, wherein the scFV is optionally linked to the effector domain via an effector linker group, such as, for example a group comprising a fluorescent protein, such as a GFP or sfGFP protein, and wherein the effector domain may further be fused to a protein destabilization domain, such as, for example, d2. Also encoded is a tag for the effector, such as, for example sfGFP (super-folder GFP), and the construct thus preferably includes a fluorescent marker.

The effector domains as encoded may comprise a chromatin modifying polypeptide selected from the group consisting of Dot1L (H3K79me2), p300 (H3K27ac), Prdm9 (H3K4me3), Kmt2b (H3K4me3), Set1a (H3K4me3), Setd2 (H3K36me3), Ring1b (H2AK119ub), Ezh2 (H3K27me3), G9a (H3K9me2), Setdb1 (H3K9me3), Suv39h1 (H3K9me3), Kmt5C (H4K20me3), Dnmt3a3L (DNAme), Ogt (GlcNAC), Prmt5 (H4R3me2s), Hdac1/2/3/4 (histone deacetylases), Sirt1/2/3/6 (histone deacetylases), Kat2a (lysine acetyltransferase), Lsd1 (H3K4me demethylase), Kdm5a/b/c (H3K4 demethylase), Kdm2b (H3K4 and H3K79 demethylase), Tet1/2/3 (methylcytosine dioxygenase), Utx (H3K27 demethylase), JMJD3 (H3K27 demethylase), Kdm4a/b/c/d (H3K36 and H3K9 demethylase) the catalytic domains (CD) thereof, the catalytic domains (CD) thereof fused to an effector domain binding motif-specific scFV domain (CDscFV) and a fragment antigen-binding (Fab) domain thereof. Also included are effectors that have the effect when combined (combinatorial effectors), which usually are co-expressed.

According to the present invention, the nucleic acid is selected from DNA, RNA, PNA or combinations thereof.

In one preferred embodiment, the set of genetic constructs is present in the form of at least one vector or plasmid, such as an expression vector, comprising the set of nucleic acids according to the present invention. Preferably, each of the nucleic acids encoding for an effector domain, CD and/or CDscFV comprises an inducible promotor, such as a tet-responsive promoter, for example a dox-responsive promoter. This allows for a fine-tuning of the expression of the effectors, and thus an additionally preferred functional control.

A key element for epigenetic editing as a biomedical strategy in humans is delivery of the complex and/or the components thereof into the relevant cells. Issues to be considered are safety, efficiency and target specificity. Several vehicles for CRISPR/Cas9 in vivo delivery have been identified. Viral delivery is the most common platform for (epi)genome therapy delivery. Lentiviral systems are widely used but suffer from problems associated with integration into the host genome. Instead, adeno-associated virus (AAV) constructs are used, which do not integrate genomically. However, AAV only have limited cargo capacity, which requires the use of smaller CRISPR/Cas9 systems, such as Staphylococcus aureus (sa) Cas9. Nanoparticles may further be used to provide an efficient and site-specific delivery of a CRISPR/Cas9 cargo in vivo. They possess a high loading capacity and good stability, as well as the potential for timely control of their cargo release. Preferred is a particle that is conjugated to nanomaterials in order to improve safety, cellular intake and specificity towards a designated cell type or tissue. Furthermore, extracellular vesicles may be used as cargo system for targeted delivery of epigenome editing molecules. Respective examples are known to the person of skill and are described in the literature. Most preferred is therefore a set of genetic constructs according to the present invention, wherein said constructs are viral constructs, for example derived from AAV, lentiviruses or retroviruses.

Included in the invention are further methods for introducing the nucleic acids and/or the genetic constructs into a cell or tissue, comprising suitably transforming or transfecting the cell or tissue with the nucleic acids and/or the genetic constructs. Methods are known in the art, and, for example, include infection or electroporation. The methods can be performed in vitro or in vivo.

Yet another aspect of the present invention then relates to a recombinant cell or tissue, comprising the set of nucleic acids according to the present invention, and/or the set of genetic constructs according to the present invention. Preferably, the recombinant cell or tissue is produced as described herein. Depending on the purpose, the cells or tissues can be prokaryotic or eukaryotic, such as bacterial or mammalian cells, e.g., human cells. Further examples are cells that are used to produce or multiply the complex according to the invention, or cells that are related to a disease-phenotype (see below) and are thus epigenetically treated using the complex according to the present invention.

Still another aspect of the present invention then relates to a method for producing the complex according to the present invention, comprising expressing the set of nucleic acids according to the present invention, and/or the set of genetic constructs according to the present invention in the recombinant cell according to the present invention, optionally comprising the step of inducing expression, for example using an antibiotic, such as a tetracycline, such as doxycycline.

Still another aspect of the present invention then relates to a method for epigenetically modifying chromatin, in particular specifically epigenetically modifying chromatin, in a cell, tissue, cellular nucleus, and/or sample comprising chromatin, comprising introducing into the cell, tissue, cellular nucleus, and/or sample the complex according to the present invention, and one or more guide RNA, thereby specifically epigenetically modifying chromatin in the cell, tissue, cellular nucleus, and/or sample. The method can be performed in vitro or in vivo. Strategies to deliver the complex according to the present invention, and one or more guide RNA as well as constructs for delivery are discussed above. Preferred is a method that epigenetically modifies cells that are related to a disease-phenotype (see below) and are thus epigenetically treated using the complex and one or more guide RNA according to the present invention.

Preferred is the method according to the present invention, wherein the epigenetic modification comprises histone methylation, DNA methylation, histone acetylation, histone ubiquitination, DNA demethylation, histone deacetylation, multiplexed epigenetic editing of histones, H3K9me2/3+DNA methylation, H3K4me3+H3K36me3, H3K4me3+H3K79me2, H3K36me3+H3K79me2, H3K9me2/3+H4K20me3, bivalent epigenetic editing of histones, and/or polycomb epigenetic editing of histones.

Further preferred is the method according to the present invention, wherein the epigenetic modification comprises a temporary induction of the expression of the complex according to the present invention and optionally the one or more guide RNA, for example using an antibiotic, such as a tetracycline, such as doxycycline. For this, preferably, each of the nucleic acids encoding for an effector domain, CD and/or CDscFV comprises an inducible promotor, such as a tet-responsive promoter, for example a dox-responsive promoter. This then allows for the temporary induction of the expression of the complex or parts thereof, in particular the effector/s, and thus an additionally preferred functional control.

Preferred is the method according to the present invention, wherein the complex comprises at least two or more, at least three or more, at least four or more, or at least five or more different effector domains, such as, for example a combination of Setd2-CD^{scFV} for H3K36me3 and Prdm9-CD^{scFV} for H3K4me3, and preferably no KRAB and/or VPR effector domain.

Still another aspect of the present invention then relates to a method for specifically epigenetically modifying chromatin in a cell, tissue, cellular nucleus, and/or sample comprising chromatin, comprising introducing into the cell, tissue, cellular nucleus, and/or sample the complex according to the present invention, and one or more guide RNA, thereby specifically epigenetically modifying chromatin in the cell, tissue, cellular nucleus, and/or sample.

As mentioned above, a major advantage of the complex according to the present invention is the possibility to specifically “tune” the chromatin modifying activity based on a specific selection and thus combination of the effector domains as used, such as, for example, a specific DNA methylation activity, a histone methylation activity, a specific histone acetylation or ubiquitination activity, and/or a specific chromatin demethylation/deacetylation activity or combinations thereof. The chromatin modification thus may comprise histone methylation, DNA methylation, histone acetylation, histone ubiquitination, DNA demethylation, histone deacetylation, multiplexed epigenetic editing of histones, H3K9me2/3+DNA methylation, H3K4me3+H3K36me3, H3K4me3+H3K79me2, H3K36me3+H3K79me2, H3K9me2/3+H4K20me3, bivalent epigenetic editing of histones, and/or polycomb epigenetic editing of histones.

Preferred is a method according to the present invention, wherein the epigenetic modification or tuning comprises a temporary induction of the expression of the complex according to the present invention and the one or more guide RNA.

Thus, preferred is a method according to the present invention, wherein the complex comprises at least two or more, at least three or more, at least four or more, or at least five or more different effector domains, such as, for example a combination of Setd2-CD^{scFV} for H3K36me3 and Prdm9-CD^{scFV} for H3K4me3, and preferably no KRAB and/or VPR effector domain.

Yet another aspect of the present invention then relates to a method for modulating the expression of at least one target DNA sequence in a cell, tissue, cellular nucleus, and/or sample comprising chromatin, said method comprising: introducing into the cell, tissue, cellular nucleus, and/or sample the complex according to the present invention, and one or more guide RNA sequence that is specific for the at least one target DNA sequence, thereby specifically epigenetically modulating the expression of at least one target DNA sequence in the cell, tissue, cellular nucleus, and/or sample.

In general, the expression of any desired target DNA sequence may be modulated, i.e. increased, decreased or stabilized, based on chromatin modification. The expression may be modulated directly, e.g., by modulating the transcription through the promotor or the accessibility thereof, or indirectly, e.g., by modulating the regulatory environment or “layers”, like splicing, enhancers or other regulatory elements. In contrast to complexes in the art, the present invention is able to provide this indirect modulation.

Preferred is the method according to the present invention, wherein the at least one target DNA sequence comprises a nucleic acid sequence that is specific for a condition and/or disease state is related to epigenetically modified chromatin, such as, for example, genetic disorders, proliferative disorders, such as cancer, immune cells that produce autoantibodies, bacterial or viral infections, protozoan infections, fragile-X syndrome, muscular dystrophy, kidney injury, cardiovascular diseases, shortened organismal lifespan, tissue aging, neurodegenerative disorders, such as Alzheimer's disease (AD), Parkinson's disease (PD), Huntington's disease (HD), Amyotrophic lateral sclerosis (ALS), drugs of abuse, including alcohol abuse disorder, epigenetic diseases, including imprinting disorders, such as Prader-Willi Syndrome

Further preferred is the method according to the present invention, wherein 2, 3, 4, 5, 6, 7, 8, or 9 target DNA sequences are modulated in the cell.

In general, any cell comprising the target DNA sequence may be used. Preferred is the method according to the present invention, wherein the cell is a stem cell, a cell in a tissue, a neuron, a post-mitotic cell, a cancer cell, or a fibroblast, and/or wherein the cell is an animal cell, such as mammalian cell, preferably human or rodent cell.

Another aspect of the present invention then relates to a method for detecting the biological effect of specifically epigenetically modifying chromatin in a cell, tissue, cellular nucleus, and/or sample comprising chromatin and/or modulating the expression of at least one target DNA sequence in a cell, tissue, cellular nucleus, and/or sample comprising chromatin, comprising performing the method according to the present invention, and detecting at least one biological effect in the cell, tissue, cellular nucleus, and/or sample comprising chromatin, wherein said biological effect is selected from the group consisting of changes in gene expression, changes in the amount of a protein, cis-genetic effects, changes in nucleic acid splicing, changes in the nuclear positioning of loci, changes in the formation and disruption of TADs, changes in the termination site, activating a promotor, repressing a promotor, changes in genetic-epigenetic interactions, functional relation between genetic variants and the epigenetic state of chromatin, changes in inherited methylation and imprinting, and linking a specific epigenetic change with a disease or cellular phenotype.

In this aspect, the complex according to the present invention can preferably be used to test or validate hits from EWAS (epigenome wide association studies) that show a specific epigenetic change is linked with a certain disease or phenotype. See, for example, Bhat B, and Jones G T (in: Data Analysis of DNA Methylation Epigenome-Wide Association Studies (EWAS): A Guide to the Principles of Best Practice. Methods Mol Biol. 2022; 2458:23-45. doi: 10.1007/978-1-0716-2140-0_2. PMID: 35103960) and the literature cited therein.

Preferred is the method according to the present invention, wherein the method is performed with a complex that allows for a tracking of the effect in the cell, tissue, cellular nucleus, and/or sample comprising chromatin, preferably in real-time.

Further preferred is the method according to the present invention, wherein the method further comprises detecting the effect of effector domains that are transcriptional activators, such as VPR or VPR^{scFV}, or transcriptional repressors, such as KRAB or KRAB^{scFV}, preferably as controls for the gRNA targeting efficiency and/or detecting a fluorescent protein, such as a GFP or sfGFP protein and/or tagBFP. The method according to the present invention may further comprise epigenetic targeted perturbation-sequencing in order to detect at least one biological effect in the cell, tissue, cellular nucleus, and/or sample comprising chromatin.

Preferably, methods according to the present invention can be at least in part, automated and/or performed in a high throughput format, i.e., can be partially or fully automated, e.g., performed fully or in part by robots. The methods according to the present invention can involve the use of computers and respective databases for performing and/or analysis of the results as obtained.

In general, any biological effect and disease or phenotype may be detected that is related to epigenetically modified chromatin, such as, for example, genetic disorders, proliferative disorders, such as cancer, immune cells that produce autoantibodies, bacterial or viral infections, protozoan infections, fragile-X syndrome, muscular dystrophy, kidney injury, cardiovascular diseases, shortened organismal lifespan, tissue aging, neurodegenerative disorders, such as Alzheimer's disease (AD), Parkinson's disease (PD), Huntington's disease (HD), Amyotrophic lateral sclerosis (ALS), drugs of abuse, including alcohol abuse disorder, epigenetic diseases, including imprinting disorders, such as Prader-Willi Syndrome.

Another aspect of the present invention then relates to a cell having a specifically epigenetically modified chromatin, produced through performing the method according to the present invention, and, optionally, isolating said cell, wherein preferably the cell is a stem cell, a neuron, a cancer cell, a cell in a tissue, a post-mitotic cell, or a fibroblast, and/or wherein the cell is an animal cell, such as mammalian cell, preferably human or rodent cell.

Another aspect of the present invention then relates to a method for identifying an agent specifically epigenetically modifying chromatin in a cell, tissue, cellular nucleus, and/or sample comprising chromatin, an agent modulating the expression of at least one target DNA sequence in a cell, tissue, cellular nucleus, and/or sample comprising chromatin and/or biological effect of specifically epigenetically modifying chromatin in a cell, tissue, cellular nucleus, and/or sample comprising chromatin, comprising performing the method according to the present invention in the presence and absence of a test agent, wherein the test agent is identified as an agent specifically epigenetically modifying chromatin in a cell, tissue, cellular nucleus, and/or sample comprising chromatin, an agent modulating the expression of at least one target DNA sequence in a cell, tissue, cellular nucleus, and/or sample comprising chromatin and/or biological effect of specifically epigenetically modifying chromatin in a cell, tissue, cellular nucleus, and/or sample comprising chromatin, if the modulation and/or biological effect in the presence of the agent differs from the modulation and/or biological effect in the absence of the agent or to a control.

The method according to the present invention seeks to identify a pharmaceutically active compound that is involved in modulating the epigenetic modification of chromatin in a cell, tissue, cellular nucleus, and/or sample comprising chromatin, preferably in a human cell. Conveniently, the format of the method can be quite flexible, the method requires a suitable combination of the components, i.e. the complex according to the invention, or a functional fragment thereof, and the at least one pharmaceutically active candidate compound, i.e. the substance that shall be screened/identified for the activity to modulate the function. The combination can be provided as a cellular system (i.e. functioning in a cell, like in mammalian cell culture), and the components can be provided recombinantly in part or fully, or the system can be in vitro, for example as an in vitro translation system that can be readily adjusted for the purposes of the present invention, if required. Examples are described in the literature and are known to the person of skill.

Preferred is a method according to the present invention, wherein said method with the at least one candidate compound is performed in vitro, in cell culture or in vivo, preferably in a non-human mammal, or comprises in silico molecular modeling, for example using a suitable computer program. Further preferred is a method according to the present invention, furthermore comprising a pre-selection step comprising molecular modeling of said binding of said at least one candidate compound to the complex or epigenetic modification site (e.g. histone) or target nucleic acid or a functional fragment thereof, for example using a computer program, such as SwissDock. That is, the present invention here includes a pre-selection based on the binding properties as modelled in silico, e.g. based on the whole or a part of the structural element, either isolated or in the context with the complex, before including compounds in the more complex full in vivo and/or vitro assays.

Methods according to the present invention can be implemented using automation (robotics), and may be performed in a high-throughput format.

The candidate compound and/or that is to be identified or screened in the context of the present invention, can be any chemical substance or any mixture thereof. Preferably, said compound is selected from a chemical substance, a substance selected from a peptide library, a library of small organic molecules, a combinatory library, a cell extract, in particular a plant cell extract, a “small molecular drug” (i.e. having a molecular weight of less than about 500 Da), a repurposable drug, a protein and/or a protein fragment, and an antibody or fragment thereof, and suitable derivatives thereof. Small molecular drugs are preferred.

Preferred is the method according to the present invention, further comprising testing of dose-dependent responses of the agent. Respective methods are known to the person of skill.

The selected or screened compound can then be modified. Said modification can take place in an additional preferred step of the methods of the invention as described herein, wherein, for example, after analyzing the binding to the complex and/or the modulation of the epigenetic modification, e.g. in the presence and absence of said compound as selected, said compound is further chemically modified as described for example, in the examples below, and analyzed again for its effect on the binding and/or modulation. Said “round of modification(s)” can be performed for one or several times in all the methods, in order to optimize the effect of the compound, for example, in order to improve its specificity for the element (e.g. complex or histones) to be bound, and/or in order to improve its specificity for the epigenetic modification to be influenced. This strategy is also termed “directed evolution” since it involves a multitude of steps including modification and selection, whereby binding compounds are selected in an “evolutionary” process optimizing its capabilities with respect to a particular property, e.g. its binding activity, its ability to activate, inhibit or modulate the activity, in particular the epigenetic modification activity.

The modification can also be simulated in silico before additional tests are performed in order to confirm or validate the effect of the modified selected or screened compound from the first round of screening. Respective software programs are known in the art and readily available for the person of skill.

Modification can further be effected by a variety of methods known in the art, which include without limitation the introduction of novel side chains or the exchange of functional groups like, for example, introduction of halogens, in particular F, Cl or Br, the introduction of lower alkyl groups, preferably having one to five carbon atoms like, for example, methyl, ethyl, n-propyl, iso-propyl, n-butyl, iso-butyl, tert-butyl, n-pentyl or iso-pentyl groups, lower alkenyl groups, preferably having two to five carbon atoms, lower alkynyl groups, preferably having two to five carbon atoms or through the introduction of, for example, a group selected from the group consisting of NH₂, NO₂, OH, SH, NH, CN, aryl, heteroaryl, COH or COOH group.

Therefore, preferred is the method according to the present invention, further comprising c) chemically modifying the candidate compound as identified (see above), and repeating the method according to the present invention in order to identify a second generation candidate compound with improved binding and/or modulating properties, and optionally d) repeating step c) at least once, twice, three times or more.

The term “functional fragment” shall mean stretches and/or regions of the peptides or polypeptides that are ultimately involved in the modulation of the epigenetic modification. These areas are generally involved in binding of the compound (modulator).

In the context of the present invention, unless indicated otherwise, the term “about” shall mean+/−10% of the value as given.

Included in the invention are further methods for producing a pharmaceutically acceptable preparation comprising providing the nucleic acids and/or the genetic constructs and cells or tissues according to the present invention and admixing or formulating said nucleic acids and/or the genetic constructs according to the present invention with at least one pharmaceutically acceptable diluent or carrier. Respective methods are known in the art. Pharmaceutically acceptable carriers or excipients include diluents (fillers, bulking agents, e.g. lactose, microcrystalline cellulose), disintegrants (e.g. sodium starch glycolate, croscarmellose sodium), binders (e.g. PVP, HPMC), lubricants (e.g. magnesium stearate), glidants (e.g. colloidal SiO₂), solvents/co-solvents (e.g. aqueous vehicle, Propylene glycol, glycerol), buffering agents (e.g. citrate, gluconates, lactates), preservatives (e.g. Na benzoate, parabens (Me, Pr and Bu), BKC), anti-oxidants (e.g. BHT, BHA, Ascorbic acid), wetting agents (e.g. polysorbates, sorbitan esters), thickening agents (e.g. methylcellulose or hydroxyethylcellulose), sweetening agents (e.g. sorbitol, saccharin, aspartame, acesulfame), flavoring agents (e.g. peppermint, lemon oils, butterscotch, etc.), humectants (e.g. propylene, glycol, glycerol, sorbitol). Other suitable pharmaceutically acceptable excipients are inter alia described in Remington's Pharmaceutical Sciences, 15^{th} Ed., Mack Publishing Co., New Jersey (1991) and Bauer et al., Pharmazeutische Technologic, 5^{th} Ed., Govi-Verlag Frankfurt (1997). The person skilled in the art knows suitable formulations for respective compounds, for example topical, and will readily be able to choose suitable pharmaceutically acceptable carriers or excipients, depending, e.g., on the formulation and administration route of the pharmaceutical composition.

Another aspect of the invention relates to a pharmaceutically acceptable preparation as produced, including the cells, nucleic acids and/or the genetic constructs according to the present invention, together with a pharmaceutically acceptable auxiliary agent or carrier.

It is to be understood that the present compound and/or a pharmaceutical composition comprising the present compound is for use to be administered to a human patient. The term “administering” means administration of a sole therapeutic agent or in combination with another therapeutic agent. It is thus envisaged that the pharmaceutical composition of the present invention are employed in co-therapy approaches, i.e. in co-administration with other medicaments or drugs and/or any other therapeutic agent which might be beneficial in the context of the methods of the present invention. Nevertheless, the other medicaments or drugs and/or any other therapeutic agent can be administered separately from the compound as selected or screened and/or compound for use, if required, as long as they act in combination (i.e. directly and/or indirectly, preferably synergistically) with the present compound as selected and/or screened.

Another important aspect of the present invention then relates to the use of the present invention in medicine (see, for example, Policarpi C, Dabin J, Hackett J A. Epigenetic editing: Dissecting chromatin function in context. Bioessays. 2021 May; 43(5):e2000316. doi: 10.1002/bies.202000316. Epub 2021 Mar. 16. PMID: 33724509). Beyond its potential for unravelling biological mechanisms, epigenetic editing is also an exciting prospect for therapeutic development. Epigenetic editing represents a precise and non-invasive strategy for combating specific disease states, and the variety of possible applications make it both complementary and, in cases, preferred over gene therapy or wild-type Cas9 genetic approaches.

In principle, epigenetic editing has a better safety profile than genetic editing because it does not alter host DNA sequence and is reversible by nature. Moreover, it relies on endogenous cis-elements to regulate target gene expression, making it a more physiological approach than gene (cDNA) delivery. For example, it facilitates switching endogenous genes ON, while also retaining the co- and post-transcriptional signals for appropriate expression regulation.

Moreover, because several genes can be targeted at the same time, multiplex epigenetic editing could be used (i) to upregulate the expression of several genes simultaneously, or (ii) to switch some genes on and others off in parallel by taking advantage of the orthogonality between CRISPR-Cas9 systems. CRISPR-based epigenetic therapy holds great promise for subsets of human diseases.

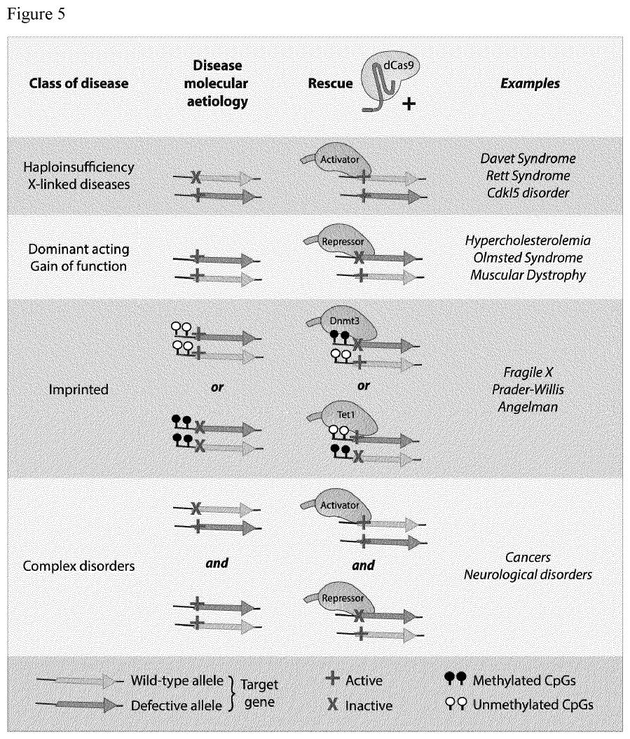

Several classes of disease represent attractive targets for development of epigenetic therapy (FIG. 5). For example, hundreds of human diseases are associated with haploinsufficiency, and selectively enhancing gene expression could compensate for the deficient gene product. This can be achieved by targeting a dCas9 fused with a transcription activation module such as VP64 and its derivatives, or with a histone modification enzyme such as p300, PRDM9 or DOT1L, as herein. In a mouse model of a severe epileptic encephalopathy called Davet syndrome, a significant attenuation of the symptoms was shown.

Other pathologies that profit from the in vivo development of the CRISPR activation system are the X-linked disorders, that arise from the expression of a faulty allele from the active X chromosome while the inactive X chromosome contains a functional copy. Rett syndrome, caused by a mutation in the MeCP2 gene, and Cdk15 disorder, are good examples of such diseases. Using programmable epigenetic editing to upregulate the expression of the functional gene copy from the inactive X chromosome in brain cells does complement the mutant allele. Epigenetic editing can potentially apply to several neurological disorders. For example, fragile X syndrome is the most prevalent form of intellectual disability in males, where abnormal DNA methylation mediates epigenetic silencing of the FMR1 gene in neurons. Interestingly, using a dCas9-Tet1 system to specifically demethylate the FMR1 promoter reversed its heterochromatic status and restored FMR1 expression in iPSC derived neurons. This strategy could be extended to imprinted disorders that arise from an aberrant DNA methylation pattern. Prader-Willi (PWS) and Angelman (AS) syndromes are good examples for the successful use of a similar approach, because some forms of PWS are due to abnormal methylation of the paternal allele, while 4% of AS results from the absence of methylation on the maternal allele. Therefore, an allele-specific therapy based on targeting of dCas9-Tet1 for the former and dCas9-Dnmt3 for the latter is promising.

In addition to targeted activation, programed gene silencing can address disease phenotypes, particularly those arising from dominant-acting genes. For example, dCas9-KRAB has been used in a mouse model of hypercholesterolemia to target the Pcsk9 gene, resulting in lower cholesterol levels in treated mice. Many inflammatory and pain-related diseases could benefit from this strategy by targeting (i) cytokines involved in the responsible inflammatory pathway, as envisioned to treat degenerative disc disease, or (ii) pain receptors located in the skin. Indeed, the skin constitutes an accessible and attractive organ for CRISPR-mediated gene silencing as it is associated with numerous monogenic and autosomal dominant disorders, such as Olmsted syndrome or the familial primary localized cutaneous amyloidosis.

Importantly, to ensure a functional gene copy remains active, allele-specific epigenetic silencing could be utilized in dominant-acting disorders. Such strategies have emerged to silence the mutant HTT allele in distinct clinical populations presenting the neurological Huntington disease. Similarly, a recent study shows that targeting the mutant DMD allele in a mouse model of Duchenne Muscular Dystrophy with the CRISPR/Cas9 system greatly improved the mouse muscle contractility.

Finally, complex diseases such as cancers could potentially profit not only from the strategies discussed above but also from multiplex epigenetic editing, by activating tumor suppressor genes and inhibiting oncogene expression at the same time. Indeed, concomitant activation and repression of different genes within the same cell has been achieved in vitro by coupling dCas9 with chemical- and light-inducible effector domains and by engineering scaffold gRNA molecules that can recruit transcriptional regulators. Moreover, the proof of concept for simultaneous gene activation has been shown in vivo in mice.

Histone methylation and aging histone methylation modifying proteins have also been shown to play a role in the regulation of organismal lifespan and tissue aging. Loss of the appropriate balance between stable and dynamic methyl marks in adult stem cells may contribute to the decline of individual tissue function with age (see Basavarajappa B S, Subbanna S. Histone Methylation Regulation in Neurodegenerative Disorders. Int J Mol Sci. 2021 Apr. 28; 22(9):4654. doi: 10.3390/ijms22094654. PMID: 33925016; PMCID: PMC8125694), e.g. in neurodegenerative disorders, such as Alzheimer's disease (AD), Parkinson's disease (PD), Huntington's disease (HD), Amyotrophic lateral sclerosis (ALS) and drugs of abuse, including alcohol abuse disorder.

The above object of the present invention is solved by a method for preventing or treating a disease related to epigenetically modified chromatin, such as, for example, genetic disorders, proliferative disorders, such as cancer, immune cells that produce autoantibodies, bacterial or viral infections, protozoan infections, fragile-X syndrome, muscular dystrophy, kidney injury, cardiovascular diseases, shortened organismal lifespan, tissue aging, neurodegenerative disorders, such as Alzheimer's disease (AD), Parkinson's disease (PD), Huntington's disease (HD), Amyotrophic lateral sclerosis (ALS), drugs of abuse, including alcohol abuse disorder, epigenetic diseases, including imprinting disorders, such as Prader-Willi Syndrome in a subject in need of such prevention or treatment, comprising administering to a subject in need of such treatment an effective amount of at least one of the complex according to the present invention, and one or more suitable guide RNA sequences, the set of nucleic acids according to the present invention, the set of genetic constructs, such as expression vectors, according to the present invention, the cell according to the present invention, and/or the agent as identified according to the present invention.

In this aspect, the complex or a nucleic acid encoding all or parts of it is used as the actual active ingredient in the prevention and/or treatment. Delivery of the complex to a patient a cell or a sample can be done in any suitable way, for example as a pharmaceutical composition comprising the isolated components of at least one complex (polypeptide and/or nucleic acid) according to the present invention together with suitable stabilizers or carriers. Another embodiment is the provision of the complex encoded on at least one nucleic acid vector to the patient, cell, tissue, sample or nucleus. These pharmaceutical compositions and uses thereof constitute preferred embodiments of the present invention. This aspect also includes the step of monitoring a treatment.

This aspect combines a diagnostic approach of the invention with a separate “regular” medical treatment, and also includes the use for monitoring the treatment. The method comprises providing a suitable treatment to said cell, tissue or organism, in particular a specific medical treatment, performing the method according to the invention as above, and modifying said treatment of said disease or medical condition based on the chromatin modification and modification of the disease as detected, e.g., when compared to a healthy control (e.g., a control based on a group of healthy or diseased samples).

By “treatment” or “treating” is meant any treatment of a disease or disorder, in a mammal, including: preventing or protecting against the disease or disorder, that is, causing, the clinical symptoms of the disease not to develop; inhibiting the disease, that is, arresting or suppressing the development of clinical symptoms; and/or relieving the disease, that is, causing the regression of clinical symptoms. By “amelioration” is meant the prevention, reduction or palliation of a state, or improvement of the state of a subject; the amelioration of a stress is the counteracting of the negative aspects of a stress. Amelioration includes, but does not require complete recovery or complete prevention of a stress.

The above object of the present invention is also solved by at least one of the complex according to the present invention, and one or more suitable guide RNA sequences, the set of nucleic acids according to the present invention, the set of genetic constructs, such as expression vectors, according to the present invention, the cell according to the present invention, and or the agent as identified according to the present invention for use in the prevention and/or treatment of diseases, or for use in the prevention and/or treatment of diseases related to epigenetically modified chromatin, such as, for example, genetic disorders, proliferative disorders, such as cancer, immune cells that produce autoantibodies, bacterial or viral infections, protozoan infections, fragile-X syndrome, muscular dystrophy, kidney injury, cardiovascular diseases, shortened organismal lifespan, tissue aging, neurodegenerative disorders, such as Alzheimer's disease (AD), Parkinson's disease (PD), Huntington's disease (HD), Amyotrophic lateral sclerosis (ALS), drugs of abuse, including alcohol abuse disorder, epigenetic diseases, including imprinting disorders, such as Prader-Willi Syndrome in a subject in need of such prevention or treatment.

Another aspect of the invention relates to the use of the complex according to the present invention, the set of nucleic acids according to the present invention, the set of genetic constructs, such as expression vectors, according to the present invention, and/or the cell according to the present invention, for specifically epigenetically modifying chromatin in a cell, tissue, cellular nucleus, and/or sample comprising chromatin according to the present invention, for modulating the expression of at least one target DNA sequence in a cell, tissue, cellular nucleus, and/or sample comprising chromatin according to the present invention, detecting the biological effect of specifically epigenetically modifying chromatin in a cell, tissue, cellular nucleus, and/or sample according to the present invention, and/or identifying an agent according to the present invention. All these aspects have been described further above.

In the context of the invention, the subject can be a mammal, preferably a mouse or human.

In another preferred aspect thereof, the object of the present invention is solved by providing a kit comprising materials for performing the methods according to the invention as a detection system, for example as a part of a diagnostic kit. The kit may also be used in a therapeutic kit, or a pharmaceutical composition comprising the isolated components of at least one complex (polypeptide and/or nucleic acid encoding it, and/or viral particles comprising these) according to the present invention together with suitable stabilizers or carriers. Another embodiment contains additional materials for the provision of the complex encoded on at least one nucleic acid vector to the subject, cell, tissue, sample or nucleus.

Preferably, said kit is provided in one or more containers, and comprises suitable enzymes, buffers, and excipients, as well as instructions for use. The components can be—at least in part—immobilized on a substrate, wherein said substrate can be exposed to said cell, tissue and/or sample.

In the context of the present invention, unless explicitly mentioned otherwise, the term “about” shall mean a value as given+/−10%.

The methods according to the present invention can be performed in vivo or in vitro, for example in an organism, a cell, tissue, and/or part thereof, like a nucleus, or in an in vitro assay, like a diagnostic assay.

Here, the inventors provide an epigenome editing platform, to systematically program specific- and combinatorial-chromatin modifications across tens-of-thousands of contexts in living cells. This resource is used to capture multi-modal functional responses at allelic, single-cell resolution, from diverse lineages. The unprecedented scale of precision perturbations will uncover the regulatory logic by which distinct chromatin modifications interact with genomic features, sequence variants, and cellular identity, to shape quantitative gene expression patterns. By integrating datasets and deploying genetic screens, the invention will further identify the trans-acting and cis-structural mechanisms that implement the functionality of chromatin marks.

A precision epigenetic editing strategy is used to define the transcriptional function and memory of heterochromatin epialleles at endogenous loci. The method of compound recruitment of multiple ‘effector’ modules using dCas9GCN4, facilitated programming of major (>10 kb) heterochromatin domains, sufficient to drive robust epigenetic silencing. These de novo domains comprised H3K9me3, H4K20me3 and DNA methylation, and concomitant loss of H3K4me3, with modification levels comparable or greater than endogenous heterochromatic regions, which are thought to self-propagate via ‘read-write’ reinforcement (see also Reinberg D, Vales L D. Chromatin domains rich in inheritance. Science. 2018 Jul. 6; 361(6397):33-34. doi: 10.1126/science.aat7871. PMID: 29976815). The inventors found that naïve pluripotent cells act as a fundamental roadblock to inheritance of heterochromatin domains occurring outside of normal genomic contexts, even when providing a selective advantage such as silencing p53.

As a preferred embodiment, the inventors have developed a modular CRISPR-based toolkit that can precisely and inducibly program—in this case—nine distinct epigenetic modifications to endogenous target loci (see FIG. 1B-H). Of these, H3K27ac and the H3K4me3 mark are usually associated with active transcription, while DNA methylation, H2AK119ub and tri-methylation of lysine 9 or 27 on histone H3 (H3K9me3 and H3K27me3, respectively) are associated with repressed chromatin regions (Berger S L et al. 2007). However, whether the observed correlations indicate causation remains unclear especially considering that the removal of some of these marks from the genome of mouse embryonic stem cells (mESCs) results only in minor transcriptional changes (Tsumura A et al. 2006; Chamberlain S J et al. 2008; Sze C C et al. 2017; Zhang T et al. 2020). Similarly, the H3K36me3 mark is found to peak within the body of active genes, yet it was shown to act as a transcriptional repressor in both yeast and mammals (Carrozza M J et al. 2005; Neri F et al. 2017). Genetic approaches (which include the genetic manipulation of the DNA sequence underlying the marks, the genetic deletion of the upstream chromatin modifying-enzymes or the mutation of specific histone residues) have been widely used to interrogate the functional relevance of epigenetic marks (Stricker S H et al 2017; Grosswendt S et al. 2020). While these approaches have been generally successful in triggering wide-spread changes in gene expression, they have not unequivocally established the causal contribution of chromatin marks per se to the observed phenotypes, due to the impossibility of distinguishing between direct and indirect effects.

Therefore, the ability to site-specifically deposit marks including H3K27me3, H3K4me3, H2AK119ub, and H3K36me3, represents a powerful gain-of-function perturbation strategy to explicitly assess their causal impact. The inventors' system or toolkit leverages dCas9 linked with a tail-array of five GCN4 motifs (dCas9^{GCN4}), each separated by a linker designed with optimal spacing to accommodate bulky proteins without sterically hindering their catalytic activity. This embodiment of dCas9^{GCN4} tethers up to five ‘effector’ proteins to a specific locus, via a GCN4-specific scFV domain (FIG. 1A). The inventors have engineered and tested a comprehensive suite of effectors, that comprise only the catalytic domain (collectively: CD^{scFV}) of chromatin-modifying enzymes, for example Setd2-CD^{scFV} for H3K36me3 and Prdm9-CD^{scFV} for H3K4me3.

The above considerations apply mutatis mutandis also to other chromatin modifying proteins or polypeptides to be used as effectors in the present invention, which may be selected from the group consisting of Dot1L (H3K79me2), p300 (H3K27ac), Prdm9 (H3K4me3), Kmt2b (H3K4me3), Set1a (H3K4me3), Setd2 (H3K36me3), Ring1b (H2AK119ub), Ezh2 (H3K27me3), G9a (H3K9me2), Setdb1 (H3K9me3), Suv39h1 (H3K9me3), Kmt5C (H4K20me3), Dnmt3a3L (DNAme), Ogt (GlcNAC), Prmt5 (H4R3me2s), Hdac1/2/3/4 (histone deacetylases), Sirt1/2/3/6 (histone deacetylases), Kat2a (lysine acetyltransferase), Lsd1 (H3K4me demethylase), Kdm5a/b/c (H3K4 demethylase), Kdm2b (H3K4 and H3K79 demethylase), Tet1/2/3 (methylcytosine dioxygenases), Utx (H3K27 demethylase), JMJD3 (H3K27 demethylase), Kdm4a/b/c/d (H3K36 and H3K9 demethylase) the catalytic domains (CD) thereof, the catalytic domains (CD) thereof fused to an effector domain binding motif-specific scFV domain (CDscFV) or a fragment antigen-binding (Fab) domain.

The epigenetic modification may comprise histone methylation, DNA methylation, histone acetylation, histone ubiquitination, DNA demethylation, histone deacetylation, multiplexed epigenetic editing of histones, H3K9me2/3+DNA methylation, H3K4me3+H3K36me3, H3K4me3+H3K79me2, H3K36me3+H3K79me2, H3K9me2/3+H4K20me3, bivalent epigenetic editing of histones, and/or polycomb epigenetic editing of histones.

The inventive system has multiple other advancements built in that collectively result in an epigenetic editing platform technology with exceptional features to enable discovery.

The attributes include:

Highly active editing. The recruitment of—in this case—five copies of a specific CD^{scFV} to a target locus greatly amplifies ON-target programming of chromatin modifications, both in amplitude and in genomic breadth. This ensures de novo histone modification deposition that is comparable to strong endogenous peaks, facilitating both negative- and positive-functional conclusions.

Catalytic domain specificity. By isolating only the catalytic cores confounding effects of targeting full-length chromatin-modifying proteins can be excluded. Since full-length proteins can have major non-catalytic functions and/or recruit other protein complexes, preferential use of catalytic-domains enables the function of targeted chromatin marks per se to be assessed.

Combinatorial epigenetic editing. The system is modular, and therefore e.g. five different CD^{scFV} can be recruited simultaneously. This enables multiplexed epigenetic editing that permits establishment of de novo domains of different chromatin modifications (e.g. bivalent or polycomb), as well as combinations.

Minimized off-targeting. Because the CD^{scFV} used here generally lack their endogenous DNA-binding domain, and because they are not directly fused to dCas9, they exhibit minimal OFF-target activity.

Temporally-resolved. Here, each CD^{scFV} is dynamically induced via a DOX-responsive promoter, and also carries a protein destabilisation (d2) domain, facilitating rapid degradation upon DOX-withdrawal. This enables interrogation of temporal responses and epigenetic memory (persistence of chromatin).

Dynamic tracking. In the embodiment, all effectors are linked with superfolder GFP (sfGFP), and all gRNAs with tagBFP, allowing the system to be tracked in real-time, for cells to be purified, and testing of dose-dependent responses (e.g. comparing GFP^{low} and GFP^{high} cells).

The inventors have generated an exhaustive set of controls. First, the inventors engineered a point-mutant for every CD^{scFV} (mut-CD^{scFV}), which specifically abrogates catalytic activity, enabling direct comparison with active CD^{scFV}. Second, as further negative controls the inventors employ recruitment of GFP^{scFV} alone, uninduced (-DOX) cells, and scrambled gRNA. Finally, as positive controls for gRNA targeting efficiency, the inventors exploit recruitment of well-characterised transcriptional activators (VPR^{scFV}) and repressors (KRAB^{scFV})

Taken together, these features permit the carefully controlled use of introducing specific chromatin modification(s) per se at any given locus, whilst ruling out confounding effects.

The present invention circumvents the central limitations of existing epigenome perturbation approaches by excluding pleiotropy and redundancy, whilst isolating functional genome x epigenome relationships. The further outcome is a deeper understanding of how specific chromatin states instruct—or reflect—gene regulation. This will aid design strategies towards precision medicine and provide guiding principles to attribute functional significance to epigenome profiles in health and disease.

Whether a defined chromatin modification at a specific locus directly affects gene expression in any given cell-type, remains essentially unknown at a quantitative level. The research pursued here will provide a systematic strategy to answer this question and thus generate fundamental insights into genome biology that addresses key challenges in the field. The unprecedent scale of precision perturbations will also provide the resources to predict functional activity of chromatin marks within defined contexts. Such modelling lays the ground work for several important advancements. Firstly, it will enable greater confidence in ascribing functional relevance to the changes observed across the vast developmental- and disease-associated epigenomics profiles generated by the community. It will, in other words, contribute to untangling cause from consequence. Secondly, the knowledge acquired here will generate rationale rules to design and optimize future epigenetic editing strategies towards application in precision medicine. For example, in guiding the design principles to induce a desirable change in the magnitude, penetrance or persistence of target gene transcription to modulate disease. Proof-of-principle of this has been demonstrated for fragile-X, muscular dystrophy, and kidney injury, and further knowledge is paramount to maximize the full therapeutic potential. Thirdly, the output will provide the means to dissect functional relationships between genetic variants and chromatin states. This prepares the foundations to mechanistically understand how human trait-associated eQTL may be modulated to exert their effects in specific tissues, disease, and across evolution. Beyond the framework of our impact expectations, the high-content readouts and unbiased screens provide ample opportunity for novel discovery of interactions and mechanisms, and therefore unexpected research avenues. There is also tremendous scope to expand the experimental strategies herein towards understanding mechanisms in genetically diverse human cells, towards engineering desirable cellular properties, and towards applications in vivo.

As stated herein, the present invention particularly relates to the following items.

Item 1. A complex comprising i) a catalytically inactive site-specific nuclease linked to ii) an array of between two and ten, preferably three to seven effector domains each having a specific chromatin modifying activity, such as, for example, a specific DNA methylation activity, a histone methylation activity, a specific histone acetylation or ubiquitination activity, and/or a specific chromatin demethylation/deacetylation activity, wherein the effector domains are each separated by a linker providing sufficient distance between the domains and the nuclease in order not to substantially interfere with their specific chromatin modifying activities, and the binding of the site-specific nuclease.

Item 2. The complex according to Item 1, wherein the catalytically inactive site-specific nuclease is selected from the group consisting of a catalytically dead (d) Cas9 from Streptococcus pyogenes, asCas12, saCas9, miniCas9, dCas9, fCas9, SceI, and dCas9/fCas9 fusions.

Item 3. The complex according to Item 1 or 2, wherein the complex comprises a fusion protein of the nuclease linked to a protein sequence comprising three to seven effector domain binding motifs that are each separated by a linker sequence, for example, Streptococcus pyogenes dCas9GCN4₍₃₋₇₎, and the complex optionally further comprising a number of effector domains, each bound to a binding motif, and the complex optionally further comprising at least one suitable guide RNA (gRNA).

Item 4. The complex according to any one of Items 1 to 3, wherein the proteinaceous complex comprises four to six effector domains, preferably five effector domains.

Item 5. The complex according to any one of Items 1 to 4, wherein the effector domain binding motif consists of an epitope comprising a peptide sequence of between 17 to 29 amino acids, preferably between 17 to 21 amino acids, and having little or no structural folding under physiological conditions, such as, for example the GCN4 epitope motif sequence.

Item 6. The complex according to any one of Items 1 to 5, wherein the length of the linker sequence is between 25 and 19 amino acids, preferably 22, further preferably comprising glycine (G) and serine (S) amino acids.

Item 7. The complex according to any one of Items 1 to 6, wherein the effector domains are bound via an effector domain binding motif-specific scFV domain, in particular a GCN4-specific scFV domain, wherein the scFV is optionally linked to the effector domain via an effector linker group, such as, for example a group comprising a fluorescent protein, such as a GFP or sfGFP protein, and wherein the effector domain may further be fused to a protein destabilization domain, such as, for example, d2.

Item 8. The complex according to any one of Items 1 to 7, wherein the effector domain comprises a chromatin modifying polypeptide selected from the group consisting of DotL (H3K79me3), p300 (H3K27ac), Prdm9 (H3K4me3), Setd2 (H3K36me3), Ring1b (H2AK119ub), Ezh2 (H3K27me3), G9a (H3K9me2), Kmt5C (H4K20me3), Dnmt3a3L (DNAme), OGT (GlcNAC), the catalytic domains (CD) thereof, the catalytic domains (CD) thereof fused to an effector domain binding motif-specific scFV domain (CDscFV).

Item 9. The complex according to any one of Items 1 to 8, wherein the chromatin modifying activity is histone methylation, such as, for example histone methylation contributing to stable or reversible gene expression control.

Item 10. The complex according to any one of Items 1 to 9, wherein the complex comprises at least two or more, at least three or more, at least four or more, or at least five or more different effector domains, such as, for example a combination of Setd2-CDscFV for H3K36me3 and Prdm9-CD^{scFV} for H3K4me3, and preferably no KRAB and/or VPR effector domain.

Item 11. A set of nucleic acids, each encoding for at least one of the proteins and/or the guide RNA (gRNA) and/or the tagBFP of the complex according to any one of Items 1 to 10.

Item 12. A set of genetic constructs, such as expression vectors, comprising the set of nucleic acids according to Item 11, wherein preferably each nucleic acid encoding for an effector domain, CD and/or CDscFV comprises an inducible promotor, such as a tet-responsive promoter, for example a dox-responsive promoter.

Item 13. The set of genetic constructs according to Item 12, wherein said constructs are viral constructs, such as viral constructs, for example derived from AAV, lentiviruses or retroviruses.

Item 14. A recombinant cell, comprising the set of nucleic acids according to Item 11, and/or the set of genetic constructs according to Item 12 or 13.

Item 15. A method for producing the complex according to any one of Items 1 to 10, comprising expressing the set of nucleic acids according to Item 11, and/or the set of genetic constructs according to Item 12 or 13 in the recombinant cell according to Item 14, optionally comprising the step of inducing expression, for example using a tetracycline.

Item 16. A method for specifically epigenetically modifying chromatin in a cell, tissue, cellular nucleus, and/or sample comprising chromatin, comprising introducing into the cell, tissue, cellular nucleus, and/or sample the complex according to any one of Items 1 to 10, and one or more guide RNA, thereby specifically epigenetically modifying chromatin in the cell, tissue, cellular nucleus, and/or sample.

Item 17. The method according to Item 16, wherein the epigenetic modification comprises histone methylation, DNA methylation, histone acetylation, histone ubiquitination, DNA demethylation, histone deacetylation, multiplexed epigenetic editing of histones, H3K9me2/3+DNA methylation, H3K4me3+H3K36me3, H3K4me3+H3K79me2, H3K36me3+H3K79me2, H3K9me2/3+H4K20me3, bivalent epigenetic editing of histones, and/or polycomb epigenetic editing of histones.

Item 18. The method according to Item 16 or 17, wherein the epigenetic modification comprises a temporary induction of the expression of the complex according to any one of Items 1 to 10 and the one or more guide RNA.

Item 19. The method according to any one of Items 16 to 18, wherein the complex comprises at least two or more, at least three or more, at least four or more, or at least five or more different effector domains, such as, for example a combination of Setd2-CD^{scFV} for H3K36me3 and Prdm9-CD^{scFV} for H3K4me3, and preferably no KRAB and/or VPR effector domain.

Item 20. A method for modulating the expression of at least one target DNA sequence in a cell, tissue, cellular nucleus, and/or sample comprising chromatin, said method comprising: introducing into the cell, tissue, cellular nucleus, and/or sample the complex according to any one of Items 1 to 10, and one or more guide RNA sequence that is specific for the at least one target DNA sequence, thereby specifically epigenetically modulating the expression of at least one target DNA sequence in the cell, tissue, cellular nucleus, and/or sample.

Item 21. The method according to Item 20, wherein the at least one target DNA sequence comprises a nucleic acid sequence that is specific for a condition and/or disease state is related to epigenetically modified chromatin, such as, for example, genetic disorders, proliferative disorders, such as cancer, immune cells that produce autoantibodies, bacterial or viral infections, protozoan infections, fragile-X syndrome, muscular dystrophy, kidney injury, cardiovascular diseases, shortened organismal lifespan, tissue aging, neurodegenerative disorders, such as Alzheimer's disease (AD), Parkinson's disease (PD), Huntington's disease (HD), Amyotrophic lateral sclerosis (ALS), drugs of abuse, including alcohol abuse disorder, epigenetic diseases, including imprinting disorders, such as Prader-Willi Syndrome

Item 22. The method according to Item 20 or 21, wherein 2, 3, 4, 5, 6, 7, 8, or 9 target DNA sequences are modulated in the cell.

Item 23. The method according to any one of Items 16 to 22, wherein the cell is a stem cell, a neuron, a post-mitotic cell, or a fibroblast, and/or wherein the cell is an animal cell, such as mammalian cell, preferably human or rodent cell.

Item 24. A method for detecting the biological effect of specifically epigenetically modifying chromatin in a cell, tissue, cellular nucleus, and/or sample comprising chromatin and/or modulating the expression of at least one target DNA sequence in a cell, tissue, cellular nucleus, and/or sample comprising chromatin, comprising performing the method according to any one of Items 16 to 23, and detecting at least one biological effect in the cell, tissue, cellular nucleus, and/or sample comprising chromatin, wherein said biological effect is selected from the group consisting of changes in gene expression, changes in the amount of a protein, cis-genetic effects, changes in nucleic acid splicing, changes in the nuclear positioning of loci, changes in the formation and disruption of TADs, changes in the termination site, activating a promotor, repressing a promotor, changes in genetic-epigenetic interactions, functional relation between genetic variants and the epigenetic state of chromatin, changes in inherited methylation and imprinting, and linking a specific epigenetic change with a disease or cellular phenotype.

Item 25. The method according to Item 24, wherein the method is performed with a complex that allows for a tracking of the effect in the cell, tissue, cellular nucleus, and/or sample comprising chromatin, preferably in real-time.

Item 26. The method according to Item 24 or 25, wherein the method further comprises detecting the effect of effector domains that are transcriptional activators, such as VPR or VPR^{scFV}, or transcriptional repressors, such as KRAB or KRAB^{scFV}, preferably as controls for the gRNA targeting efficiency and/or detecting a fluorescent protein, such as a GFP or sfGFP protein and/or tagBFP.

Item 27. The method according to any one of Items 24 to 26, wherein the method further comprises epigenetic targeted perturbation-sequencing in order to detect at least one biological effect in the cell, tissue, cellular nucleus, and/or sample comprising chromatin.

Item 28. The method according to any one of Items 16 to 27, wherein the method is, at least in part, automated and/or performed in a high throughput format.

Item 29. The method according to any one of Items 16 to 28, wherein the disease or phenotype is related to epigenetically modified chromatin, such as, for example, genetic disorders, proliferative disorders, such as cancer, immune cells that produce autoantibodies, bacterial or viral infections, protozoan infections, fragile-X syndrome, muscular dystrophy, kidney injury, cardiovascular diseases, shortened organismal lifespan, tissue aging, neurodegenerative disorders, such as Alzheimer's disease (AD), Parkinson's disease (PD), Huntington's disease (HD), Amyotrophic lateral sclerosis (ALS), drugs of abuse, including alcohol abuse disorder, epigenetic diseases, including imprinting disorders, such as Prader-Willi Syndrome.

Item 30. A cell having a specifically epigenetically modified chromatin, produced by performing the method according to any one of Items 16 to 19, and, optionally, isolating said cell, wherein preferably the cell is a stem cell, a neuron, a post-mitotic cell, or a fibroblast, and/or wherein the cell is an animal cell, such as mammalian cell, preferably human or rodent cell.

Item 31. A method for identifying an agent specifically epigenetically modifying chromatin in a cell, tissue, cellular nucleus, and/or sample comprising chromatin, an agent modulating the expression of at least one target DNA sequence in a cell, tissue, cellular nucleus, and/or sample comprising chromatin and/or biological effect of specifically epigenetically modifying chromatin in a cell, tissue, cellular nucleus, and/or sample comprising chromatin, comprising performing the method according to any one of Items 16 to 29 in the presence and absence of a test agent, wherein the test agent is identified as an agent specifically epigenetically modifying chromatin in a cell, tissue, cellular nucleus, and/or sample comprising chromatin, an agent modulating the expression of at least one target DNA sequence in a cell, tissue, cellular nucleus, and/or sample comprising chromatin and/or biological effect of specifically epigenetically modifying chromatin in a cell, tissue, cellular nucleus, and/or sample comprising chromatin, if the modulation and/or biological effect in the presence of the agent differs from the modulation and/or biological effect in the absence of the agent or to a control.

Item 32. The method according to Item 30, wherein the test agent is selected from the group consisting of a chemical molecule, a molecule selected from a library of small organic molecules, a molecule selected from a combinatory library, a cell extract, in particular a plant cell extract, a small molecular drug, a protein, a protein fragment, a molecule selected from a peptide library, and an antibody or fragment thereof.

Item 33. The method according to Item 31 or 32, further comprising testing of dose-dependent responses of the agent.

Item 34. A method for preventing or treating a disease related to epigenetically modified chromatin, such as, for example, genetic disorders, proliferative disorders, such as cancer, immune cells that produce autoantibodies, bacterial or viral infections, protozoan infections, fragile-X syndrome, muscular dystrophy, kidney injury, cardiovascular diseases, shortened organismal lifespan, tissue aging, neurodegenerative disorders, such as Alzheimer's disease (AD), Parkinson's disease (PD), Huntington's disease (HD), Amyotrophic lateral sclerosis (ALS), drugs of abuse, including alcohol abuse disorder, epigenetic diseases, including imprinting disorders, such as Prader-Willi Syndrome in a subject in need of such prevention or treatment, comprising administering to a subject in need of such treatment an effective amount of at least one of the complex according to any one of Items 1 to 10, and one or more suitable guide RNA sequences, the set of nucleic acids according to Item 11, the set of genetic constructs, such as expression vectors, according to Item 12 or 13, the cell according to Item 30, and/or the agent as identified according to any one of Items 31 to 33.

Item 35. At least one of the complex according to any one of Items 1 to 10, and one or more suitable guide RNA sequences, the set of nucleic acids according to Item 11, the set of genetic constructs, such as expression vectors, according to Item 12 or 13, the cell according to Item 30, and or the agent as identified according to any one of Items 31 to 33 for use in the prevention and/or treatment of diseases, or for use in the prevention and/or treatment of diseases related to epigenetically modified chromatin, such as, for example, genetic disorders, proliferative disorders, such as cancer, immune cells that produce autoantibodies, bacterial or viral infections, protozoan infections, fragile-X syndrome, muscular dystrophy, kidney injury, cardiovascular diseases, shortened organismal lifespan, tissue aging, neurodegenerative disorders, such as Alzheimer's disease (AD), Parkinson's disease (PD), Huntington's disease (HD), Amyotrophic lateral sclerosis (ALS), drugs of abuse, including alcohol abuse disorder, epigenetic diseases, including imprinting disorders, such as Prader-Willi Syndrome in a subject in need of such prevention or treatment.

Item 36. Use of the complex according to any one of Items 1 to 10, the set of nucleic acids according to Item 11, the set of genetic constructs, such as expression vectors, according to Item 12 or 13, and/or the cell according to Item 30, for specifically epigenetically modifying chromatin in a cell, tissue, cellular nucleus, and/or sample comprising chromatin according to any one of Items 16 to 19 and 29, for modulating the expression of at least one target DNA sequence in a cell, tissue, cellular nucleus, and/or sample comprising chromatin according to any one of Items 20 to 23 and 28, detecting the biological effect of specifically epigenetically modifying chromatin in a cell, tissue, cellular nucleus, and/or sample according to any one of Items 24 to 27, and/or identifying an agent according to any one of Items 30 to 32.

The present invention will now be further described in the following examples with reference to the accompanying Figures, nevertheless, without wanting to be limited thereto. For the purposes of the present invention, all references as cited herein are incorporated by reference in their entireties.

The present disclosure includes as sequence listing comprising SEQ ID No: 1 as part of the description, which is also incorporated by reference in its entirety.

FIG. 1A shows a schematic overview of a preferred embodiment of the complex according to the present invention. FIG. 1B-H show the quantitative enrichment of seven chromatin modifications targeted to Hbby by DOX-induction of the relevant CD^{scFV}. Importantly, levels are comparable to endogenous positive controls (high ON-target activity), whilst non-targeted loci are largely unaffected (low OFF-target activity). This is further represented in FIG. 1I for H3K4me3 and H2AK119ub following targeting with a single gRNA.

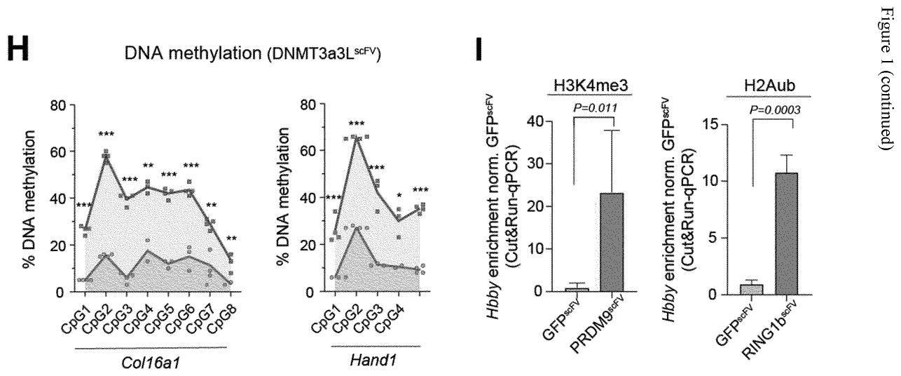

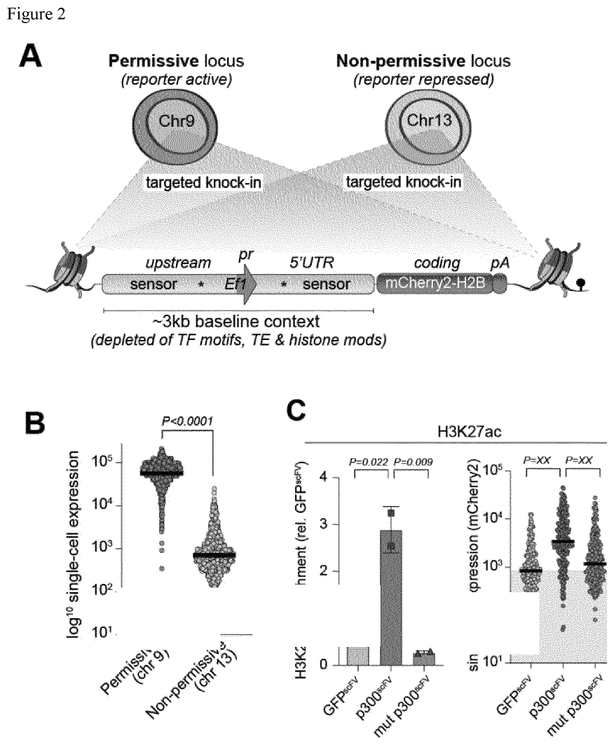

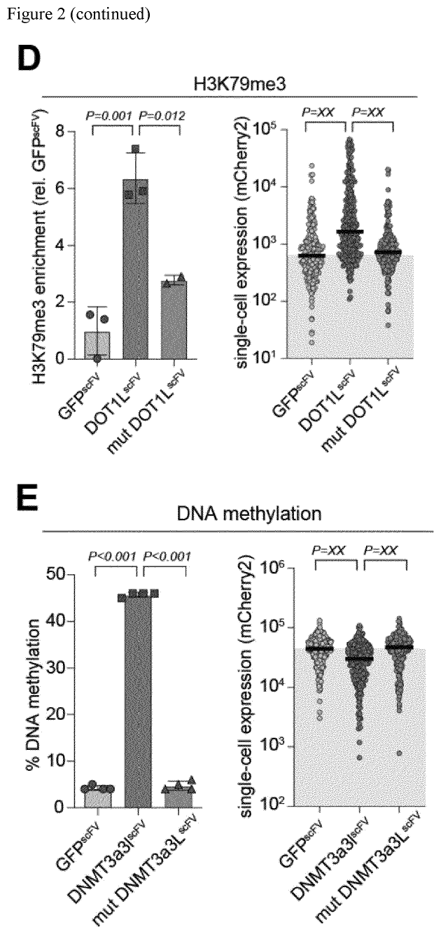

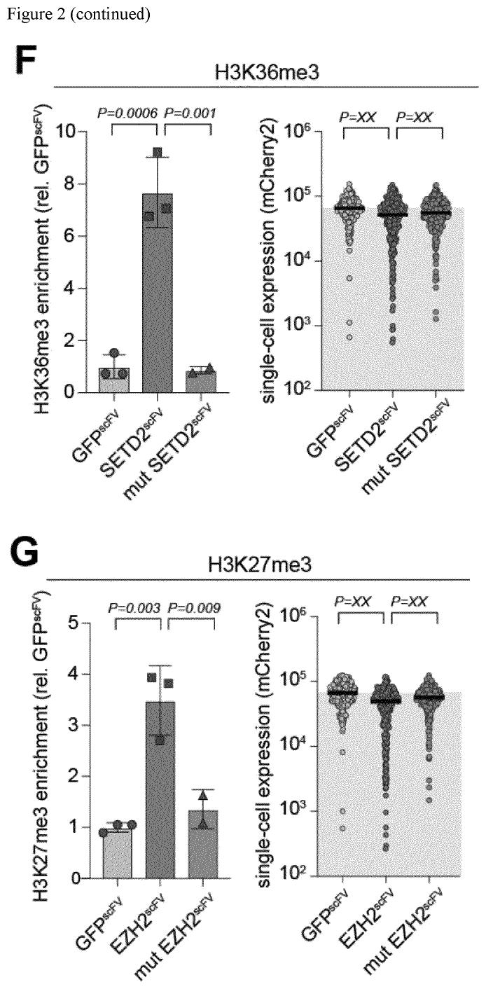

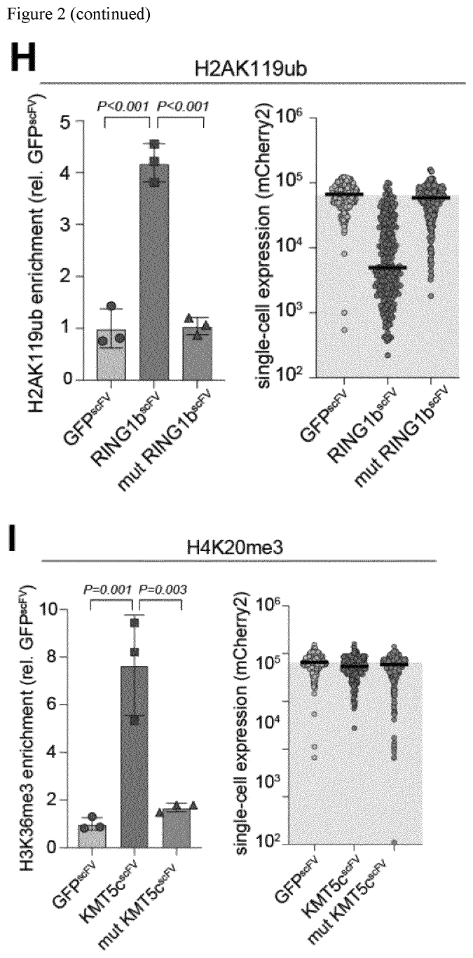

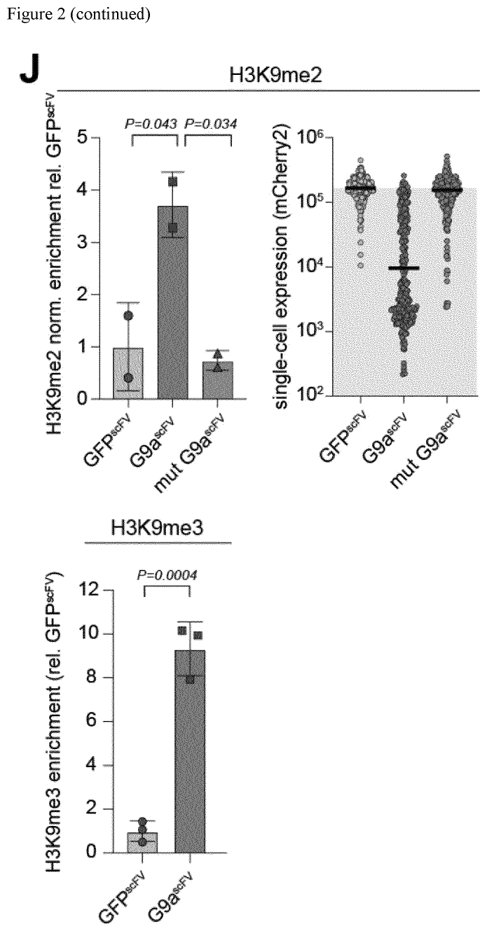

FIG. 2 shows functional responses to epigenetic editing at single-cell resolution. For example, programming H2AK119ub (FIG. 2H) to the promoter of an active reporter gene with Ring1b-CD^{scFv} (PRC1), but not its catalytic-mutant, robustly instructed transcriptional repression amongst most cells. On the other hand, targeting H3K27me3 (PRC2, FIG. 2G) with Ezh2-FL^{scFv} exhibited a partially-penetrant transcriptional response, with many cells failing to exhibit repression, indicating H3K27me3 has less instructive power at this locus.

FIG. 3 A-C show that A) combinatorial epigenetic editing of H3K27me3 and H2AK119ub reveals a synergistic effect on repressing a reporter in single-cells. B, C) Genetic-epigenetic interactions. Introducing a short MYC, OTX (3B) or CTCF (3C) motif into a reporter has minimal effects on the starting state, but specifically modulates the impact of de novo H3K27ac and H3K36me3, respectively.

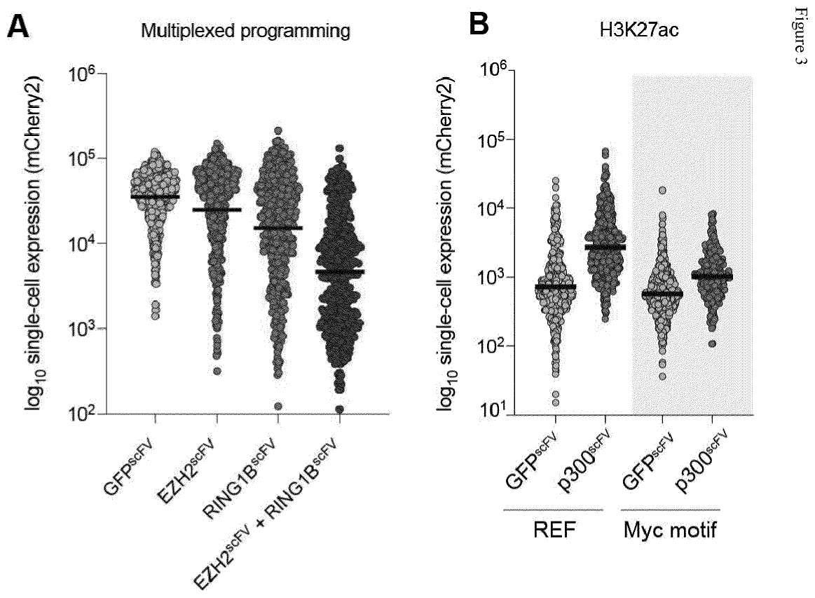

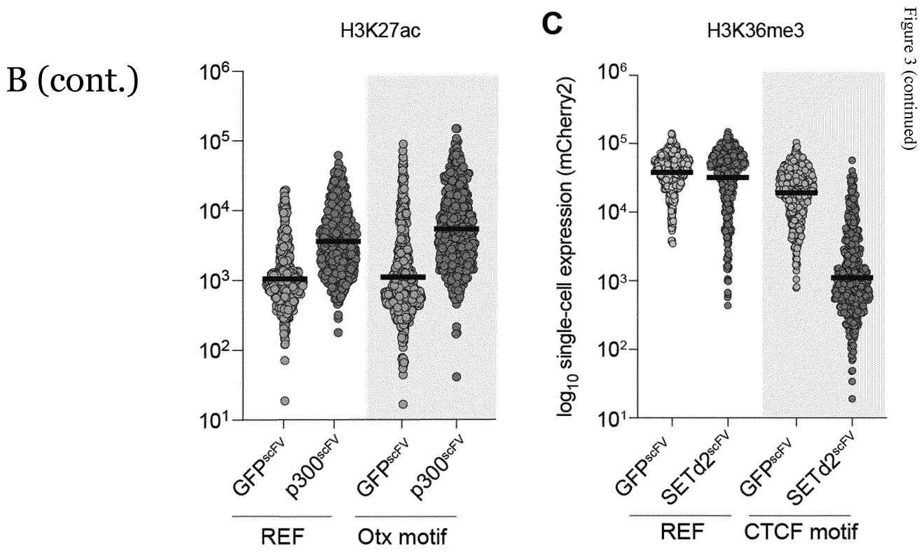

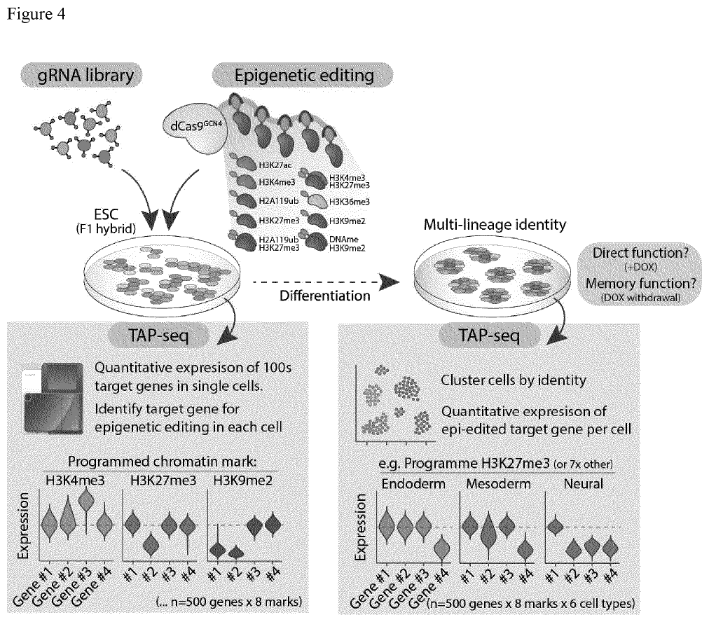

FIG. 4 shows a schematic overview of the epiTAP-seq workflow according to the invention.

FIG. 5 shows potential therapeutic applications of epigenome editing. Various classes of disease could benefit from the development of distinct CRISPR/Cas9-driven epigenome editing strategies. Shown here are examples of how mutant or wild-type alleles could be manipulated in specific disease contexts. The molecular aetiology underlying each disease type, as well as the dCas9-based rescue strategy, are shown. Figure taken from Policarpi et al. 2021.

### EXAMPLES

Epigenetic editing tools comprising dCas9^{GCN4} and all CD^{scFV} and FL^{scFV} effectors were cloned into PiggyBac recipient plasmids by homology arm recombination using In-fusion HD-Cloning (Takara #639650) according to manufacturer's instructions. The Streptococcus pyogenes dCas9^{GCN4} was PCR amplified from the PlatTET-gRNA2 plasmid (Morita et al, 2016; Addgene #82559), and cloned under the control of a TRE3G promoter in a PiggyBac backbone vector also containing the TET-ON3G transactivator and the hygromycin resistance gene driven by the EF-1a promoter.

For all effector plasmids, the scFv domain and the sfGFP coding sequence were amplified from the PlatTET-gRNA2 plasmid (Addgene #82559) and fused in frame with the catalytic domain (CD) or the full-length version (FL) of mouse Prdm9, P300, Dot1L, G9a, Kmt5c, Setd2, Ezh2 and Ring1b, all amplified from cDNA samples. Dnmt3a CD and the C-terminal part of mouse Dnmt3L (3a3L) were instead amplified from pET28-Dnmt3a3L-sc27 (Addgene #71827). The resulting constructs were cloned in PiggyBac plasmids under the control of the TRE3G promoter. These vectors also carry constitutive expression of a Neomycin resistance gene. The control GFPscFv effector was cloned as described above but lacks any chromatin modifying domain. Finally, catalytic mutant (mut-CD^{scFV}) effectors, were also cloned as described above. Specific mutations that abolish the catalytic activity were introduced during PCR amplification of the cDNA/plasmid template by mean of oligonucleotide primers designed with mismatching nucleotides.

The guide RNA plasmid, carrying an enhanced gRNA scaffold, was amplified from Addgene plasmid #60955 and cloned into a PiggyBac recipient vector also constitutively expressing a Puromycin resistance gene and TagBFP.

For designing all gRNAs used to target the epigenetic editing system, the GPP web portal (Broad Institute) was employed. gRNA forward and reverse strands carrying appropriate overhangs (10 μM final concentration) were annealed in annealing buffer containing 10 mM Tris, pH 7.5-8.0, 60 mM NaCl, 1 mM EDTA, at 95° C. for 3 min and allowed to cool down at RT for >30 min. Annealed gRNAs were ligated with T4-DNA ligase (NEB #M0202S) for 1 h at 37° C. into the PiggyBac recipient vector previously digested with BlpI (NEB #R0585S) and BstXI (NEB #R0113S) restriction enzymes. Final plasmids were amplified by bacteria transformation and purified by endotoxin-free midi-preparations (ZymoResearch #D4200). Correct assembly and sequences were confirmed by Sanger sequencing (Azenta).

### Results

In the context of the present invention, the inventors have confirmed that the present system is capable of specific and highly-efficient ON-target epigenetic editing at an endogenous locus.

FIG. 1B-H show the quantitative enrichment of seven chromatin modifications targeted to Hbby by DOX-induction of the relevant CD^{scFV}. Importantly, levels are comparable to endogenous positive controls (high ON-target activity), whilst non-targeted loci are largely unaffected (low OFF-target activity). This is further represented in FIG. 1I for H3K4me3 and H2AK119ub following targeting with a single gRNA.

The inventors have further examined aspects of chromatin function using a reporter system, knocked-in to two specific genomic locations. They found strong and specific enrichment of the expected histone modification by each CD^{scFV}, but no enrichment when the mut-CD^{scFV} or GFP^{scFV} is targeted. Exploiting reporter activity, they were able to reproducibly detect quantitative functional responses to epigenetic editing at single-cell resolution (FIG. 2). For example, programming H2AK119ub to the promoter with Ring1b-CD^{scFV} (PRC1), but not its catalytic-mutant, robustly instructed transcriptional repression amongst most cells. On the other hand, targeting H3K27me3 (PRC2) with Ezh2-FL^{scFV} exhibited a partially-penetrant transcriptional response, with many cells failing to exhibit repression, indicating H3K27me3 has less instructive power at this locus.

Multiplexed programming of H2AK119ub and H3K27me3 led to a synergistic effect, with penetrant silencing quantitatively beyond effects of either mark individually (FIG. 3A). Examination of further chromatin modifications, such as H3K4me3 and H3K9me2/3 revealed distinct functionalities at the reporter locus, whilst others such as H4K20me3 and H3K36me3 did not elicit transcriptional responses.

Taken together, these data support precise, highly efficient capacity to program a panel of chromatin modifications to a target, and reveal single-cell transcriptional responses at the single test locus in ESC.

The inventors have further investigated genetic-epigenetic interactions within our synthetic system. For example, we find that whilst programming H3K27ac activates a repressed reporter, experimentally inserting a short 10nt MYC motif (E-box) into the same reporter attenuates the effect (FIG. 3B). Conversely, introducing a 9nt OTX2 motif amplifies the transcriptional impact of H3K27ac. The inventors found that inserting short CTCF motifs led to a behavior-switch of H3K36me3 targeted to the promoter, from neutral (non-functional) to strongly transcriptionally repressive (FIG. 3C).

These data demonstrate robust quantitative interactions between genetic motifs and causal epigenomic functionality. More generally, such results using a reductionist reporter strategy highlight the power of precise epigenetic perturbations to detect quantitative responses, and cis-genetic influences.

To further address the context-dependent principles of chromatin function, the inventors propose to develop epigenetic targeted perturbation-sequencing (epiTAP-seq). This strategy will enable multi-dimensional evaluation of the function of numerous chromatin states. The principle builds on TAP-seq (Schraivogel, D., et al. Targeted Perturb-seq enables genome-scale genetic screens in single cells. 17, 629-635. Nat Methods (2020)), which derives quantitative expression changes in single-cells in response to guided perturbation(s) (FIG. 4). By intersecting this approach with our suite of chromatin perturbations, and focusing on the direct target, rather than network response, we can measure transcriptional consequences at endogenous genes at an unprecedented scale. The complexity afforded by programming twelve epigenetic marks and combinations to a broad range of genes, as well as leveraging cis-genetic variants, and multiple cell types, produces tens of thousands of contexts to dissect causal relationships.

This enables interrogation of a large number of key questions. For example what is the precise nature of transcriptional response to a de novo chromatin mark (quantitation), the functional penetrance of modification(s) between single cells, or between different genes (robustness), the quantitative outcomes of diverse genetic-epigenetic interactions at the level of genomic features or cis-variants (context-dependency), the impact of cellular environment on regulatory response (cell-type specificity) and/or, the extent of epigenetic and transcriptional persistence of chromatin states (memory). Overall, by implementing epiTAP-seq into the system and method according to the present invention and integrating the data with a complementary range of unbiased screens and epigenomic profiling approaches, we are uniquely positioned to exploit the opportunities afforded by precision epigenetic editing to dissect complex genome regulatory mechanisms in a controlled and comprehensive manner.

### LITERATURE AS CITED

1 Grosswendt, S., Kretzmer, H., Smith, Z. D., Kumar, A. S., Hetzel, S., Wittler, L., Klages, S., Timmermann, B., Mukherji, S. & Meissner, A. Epigenetic regulator function through mouse gastrulation. 584, 102-108. Nature (2020).
2 De Santa, F., Totaro, M. G., Prosperini, E., Notarbartolo, S., Testa, G. & Natoli, G. The histone H3 lysine-27 demethylase Jmjd3 links inflammation to inhibition of polycomb-mediated gene silencing. 130, 1083-1094. Cell (2007).
3 Cavalli, G. & Heard, E. Advances in epigenetics link genetics to the environment and disease. 571, 489-499. Nature (2019).
4 Roadmap Epigenomics, C. et al. Integrative analysis of 111 reference human epigenomes. 518, 317-330. Nature (2015).
5 Moore, J. E., Purcaro, M. J., Pratt, H. E., Epstein, C. B., Shoresh, N., Adrian, J., Kawli, T., Davis, C. A., Dobin, A. & Kaul, R. Expanded encyclopaedias of DNA elements in the human and mouse genomes. 583, 699-710. Nature (2020).
6 Stunnenberg, H. G., International Human Epigenome, C. & Hirst, M. The International Human Epigenome Consortium: A Blueprint for Scientific Collaboration and Discovery. 167, 1897. Cell (2016).
7 Allis, C. D. & Jenuwein, T. The molecular hallmarks of epigenetic control. 17, 487-500. Nat Rev Genet (2016).
8 Schuettengruber, B., Bourbon, H. M., Di Croce, L. & Cavalli, G. Genome Regulation by Polycomb and Trithorax: 70 Years and Counting. 171, 34-57. Cell (2017).
9 Greenberg, M. V. C. & Bourc'his, D. The diverse roles of DNA methylation in mammalian development and disease. 20, 590-607. Nat Rev Mol Cell Biol(2019).
10 Dorighi, K. M., Swigut, T., Henriques, T., Bhanu, N. V., Scruggs, B. S., Nady, N., Still, C. D., 2nd, Garcia, B. A., Adelman, K. & Wysocka, J. M113 and M114 Facilitate Enhancer RNA Synthesis and Transcription from Promoters Independently of H3K4 Monomethylation. 66, 568-576 e564. Molecular cell (2017).
11 Zhang, T., Zhang, Z., Dong, Q., Xiong, J. & Zhu, B. Histone H3K27 acetylation is dispensable for enhancer activity in mouse embryonic stem cells. 21, 45. Genome Biol (2020).
12 Nakamura, M., Gao, Y., Dominguez, A. A. and Qi, L. S. CRISPR technologies for precise epigenome editing. 23, 11-22. Nat Cell Biol (2021).
13 Policarpi, C., Dabin, J. and Hackett, J. A. Epigenetic editing: Dissecting chromatin function in context. 43, e2000316. Bioessays (2021).
14 Liu, X. S., Wu, H., Krzisch, M., Wu, X., Graef, J., Muffat, J., Hnisz, D., Li, C. H., Yuan, B., Xu, C., Li, Y., Vershkov, D., Cacace, A., Young, R. A. & Jaenisch, R. Rescue of Fragile X Syndrome Neurons by DNA Methylation Editing of the FMR1 Gene. Cell (2018).
15 Consortium, G. T. The GTEx Consortium atlas of genetic regulatory effects across human tissues. 369, 1318-1330. Science (2020).
16 Maurano, M. T. et al. Systematic localization of common disease-associated variation in regulatory DNA. 337, 1190-1195. Science (2012).
17 Izzo, F. et al. DNA methylation disruption reshapes the hematopoietic differentiation landscape. Nat Genet (2020).
18 Lambert, S. A., Jolma, A., Campitelli, L. F., Das, P. K., Yin, Y., Albu, M., Chen, X., Taipale, J., Hughes, T. R. & Weirauch, M. T. The Human Transcription Factors. 172, 650-665. Cell (2018).
19 Hanna, R. E., Hegde, M., Fagre, C. R., DeWeirdt, P. C., Sangree, A. K., Szegletes, Z., Griffith, A., Feeley, M. N., Sanson, K. R., Baidi, Y., Koblan, L. W., Liu, D. R., Neal, J. T. & Doench, J. G. Massively parallel assessment of human variants with base editor screens. 184, 1064-1080 e1020. Cell (2021).
20 Carlini, V., Policarpi, C. & Hackett, J. A. Epigenetic inheritance is gated by naive pluripotency and Dppa2, e108677. EMBO J (2022).
21 Przybyla, L. & Gilbert, L. A. A new era in functional genomics screens. Nat Rev Genet (2021).
22 Adamson, B. et al. A Multiplexed Single-Cell CRISPR Screening Platform Enables Systematic Dissection of the Unfolded Protein Response. 167, 1867-1882 e1821. Cell(2016).
23 Dixit, A., Parnas, O., Li, B., Chen, J., Fulco, C. P., Jerby-Arnon, L., Marjanovic, N. D., Dionne, D., Burks, T., Raychowdhury, R., Adamson, B., Norman, T. M., Lander, E. S., Weissman, J. S., Friedman, N. & Regev, A. Perturb-Seq: Dissecting Molecular Circuits with Scalable Single-Cell RNA Profiling of Pooled Genetic Screens. 167, 1853-1866 e1817. Cell (2016).
24 Datlinger, P., Rendeiro, A. F., Schmidl, C., Krausgruber, T., Traxler, P., Klughammer, J., Schuster, L. C., Kuchler, A., Alpar, D. and Bock, C. Pooled CRISPR screening with single-cell transcriptome readout. 14, 297-301. Nat Methods (2017).
25 Gasperini, M., Hill, A. J., McFaline-Figueroa, J. L., Martin, B., Kim, S., Zhang, M. D., Jackson, D., Leith, A., Schreiber, J., Noble, W. S., Trapnell, C., Ahituv, N. & Shendure, J. A Genome-wide Framework for Mapping Gene Regulation via Cellular Genetic Screens. 176, 1516. Cell (2019).
26 Schraivogel, D., Gschwind, A. R., Milbank, J. H., Leonce, D. R., Jakob, P., Mathur, L., Korbel, J. O., Merten, C. A., Velten, L. and Steinmetz, L. M. Targeted Perturb-seq enables genome-scale genetic screens in single cells. 17, 629-635. Nat Methods (2020).
   Additional literature as cited
Berger, S. The complex language of chromatin regulation during transcription. Nature 447, 407-412 (2007).
Tsumura A, Hayakawa T, Kumaki Y, Takebayashi S, Sakaue M, Matsuoka C, Shimotohno K, Ishikawa F, Li E, Ueda H R, Nakayama J, Okano M. Maintenance of self-renewal ability of mouse embryonic stem cells in the absence of DNA methyltransferases Dnmt1, Dnmt3a and Dnmt3b. Genes Cells. 2006 July; 11(7):805-14.
Chamberlain, S. J., Yee, D., and Magnuson, T. (2008). Polycomb repressive complex 2 is dispensable for maintenance of embryonic stem cell pluripotency. Stem Cells 26, 1496-1505.
Sze C C, Cao K, Collings C K, et al. Histone H3K4 methylation-dependent and-independent functions of Set1A/COMPASS in embryonic stem cell self-renewal and differentiation. Genes Dev. 2017; 31(17):1732-1737.
Zhang, T., Zhang, Z., Dong, Q. et al. Histone H3K27 acetylation is dispensable for enhancer activity in mouse embryonic stem cells. Genome Biol 21, 45 (2020).
Carrozza M J, Li B, Florens L, Suganuma T, Swanson S K, Lee K K, Shia W J, Anderson S, Yates J, Washburn M P, Workman J L. Histone H3 methylation by Set2 directs deacetylation of coding regions by Rpd3S to suppress spurious intragenic transcription. Cell. 2005 Nov. 18; 123(4):581-92.
Neri F, Rapelli S, Krepelova A, Incarnato D, Parlato C, Basile G, Maldotti M, Anselmi F, Oliviero S. Intragenic DNA methylation prevents spurious transcription initiation. Nature. 2017 Mar. 2; 543(7643):72-77.
Stricker S H, Köferle A, Beck S. From profiles to function in epigenomics. Nat Rev Genet. 2017 January; 18(1):51-66.
Grosswendt S, Kretzmer H, Smith Z D, Kumar A S, Hetzel S, Wittler L, Klages S, Timmermann B, Mukherji S, Meissner A. Epigenetic regulator function through mouse gastrulation. Nature. 2020 August; 584(7819):102-108.

1. A complex comprising:
i) a catalytically inactive site-specific nuclease, linked to
ii) an array of between two and ten effector domains each having a specific chromatin modifying activity,
wherein the effector domains are each separated by a linker providing sufficient distance between the domains and the nuclease in order not to substantially interfere with their specific chromatin modifying activities, and the binding of the site-specific nuclease.

2. The complex according to claim 1, wherein the complex comprises a fusion protein of the nuclease linked to a protein sequence comprising three to seven effector domain binding motifs that are each separated by a linker sequence, and the complex optionally further comprising a number of effector domains, each bound to a binding motif, and the complex optionally further comprising at least one guide RNA (gRNA).

3. The complex according to claim 1, wherein the length of the linker sequence is between 25 and 19 amino acids.

4. The complex according to claim 1, wherein the effector domains are bound via a GCN4-specific scFV domain, wherein the scFV is optionally linked to the effector domain via an effector linker group.

5. The complex according to claim 1, wherein the effector domain comprises a chromatin modifying polypeptide selected from the group consisting of Dot1L (H3K79me2), p300 (H3K27ac), Prdm9 (H3K4me3), Kmt2b (H3K4me3), Set1a (H3K4me3), Setd2 (H3K36me3), Ring1b (H2AK119ub), Ezh2 (H3K27me3), G9a (H3K9me2), Setdb1 (H3K9me3), Suv39h1 (H3K9me3), Kmt5C (H4K20me3), Dnmt3a3L (DNAme), Ogt (GlcNAC), Prmt5 (H4R3me2s), Hdac1/2/3/4 (histone deacetylases), Sirt1/2/3/6 (histone deacetylases), Kat2a (lysine acetyltransferase), Lsd1 (H3K4me demethylase), Kdm5a/b/c (H3K4 demethylase), Kdm2b (H3K4 and H3K79 demethylase), Tet1/2/3 (methylcytosine dioxygenase), Utx (H3K27 demethylase), JMJD3 (H3K27 demethylase), Kdm4a/b/c/d (H3K36 and H3K9 demethylase), the catalytic domains (CD) thereof, the catalytic domains (CD) thereof fused to an effector domain binding motif-specific scFV domain (CDscFV) and a fragment antigen-binding (Fab) domain thereof.

6. A set of nucleic acids, each encoding at least one of the proteins of the complex according to claim 1.

7. A set of genetic constructs comprising the set of nucleic acids according to claim 6.

8. A recombinant cell, comprising the set of nucleic acids according to claim 6.

9. A method for specifically epigenetically modifying chromatin in a cell, tissue, cellular nucleus, and/or sample comprising chromatin, comprising introducing into the cell, tissue, cellular nucleus, and/or sample the complex according to claim 1, and one or more guide RNA, thereby specifically epigenetically modifying chromatin in the cell, tissue, cellular nucleus, and/or sample.

10. The method according to claim 9, wherein the epigenetic modification comprises histone methylation, DNA methylation, histone acetylation, histone ubiquitination, DNA demethylation, histone deacetylation, multiplexed epigenetic editing of histones, H3K9me2/3+DNA methylation, H3K4me3+H3K36me3, H3K4me3+H3K79me2, H3K36me3+H3K79me2, H3K9me2/3+H4K20me3, bivalent epigenetic editing of histones, and/or polycomb epigenetic editing of histones.

11. A method for modulating the expression of at least one target DNA sequence in a cell, tissue, cellular nucleus, and/or sample comprising chromatin, said method comprising:
introducing into the cell, tissue, cellular nucleus, and/or sample the complex according to claim 1, and one or more guide RNA sequence that is specific for the at least one target DNA sequence, thereby specifically epigenetically modulating the expression of at least one target DNA sequence in the cell, tissue, cellular nucleus, and/or sample, wherein preferably the at least one target DNA sequence comprises a nucleic acid sequence that is specific for a condition and/or disease state is related to epigenetically modified chromatin, such as, for example, genetic disorders, proliferative disorders, such as cancer, immune cells that produce autoantibodies, bacterial or viral infections, protozoan infections, fragile-X syndrome, muscular dystrophy, kidney injury, cardiovascular diseases, shortened organismal lifespan, tissue aging, neurodegenerative disorders, such as Alzheimer's disease (AD), Parkinson's disease (PD), Huntington's disease (HD), Amyotrophic lateral sclerosis (ALS), drugs of abuse, including alcohol abuse disorder, epigenetic diseases, including imprinting disorders, such as Prader-Willi Syndrome, wherein more preferably 2, 3, 4, 5, 6, 7, 8, or 9 target DNA sequences are modulated in the cell.

12. A method for detecting the biological effect of specifically epigenetically modifying chromatin in a cell, tissue, cellular nucleus, and/or sample comprising chromatin and/or modulating the expression of at least one target DNA sequence in a cell, tissue, cellular nucleus, and/or sample comprising chromatin, comprising performing the method according to claim 9, and detecting at least one biological effect in the cell, tissue, cellular nucleus, and/or sample comprising chromatin, wherein said biological effect is selected from the group consisting of changes in gene expression, changes in the amount of a protein, cis-genetic effects, changes in nucleic acid splicing, changes in the nuclear positioning of loci, changes in the formation and disruption of TADs, changes in the termination site, activating a promotor, repressing a promotor, changes in genetic-epigenetic interactions, functional relation between genetic variants and the epigenetic state of chromatin, changes in inherited methylation and imprinting, and linking a specific epigenetic change with a disease or cellular phenotype.

13. A cell having a specifically epigenetically modified chromatin, produced by performing the method according to claim 9, and, optionally, isolating said cell, wherein the cell is a stem cell, a neuron, a post-mitotic cell, or a fibroblast.

14. A method for identifying an agent specifically epigenetically modifying chromatin in a cell, tissue, cellular nucleus, and/or sample comprising chromatin, an agent modulating the expression of at least one target DNA sequence in a cell, tissue, cellular nucleus, and/or sample comprising chromatin and/or biological effect of specifically epigenetically modifying chromatin in a cell, tissue, cellular nucleus, and/or sample comprising chromatin, comprising performing the method according to claim 9 in the presence and absence of a test agent, wherein the test agent is identified as an agent specifically epigenetically modifying chromatin in a cell, tissue, cellular nucleus, and/or sample comprising chromatin, an agent modulating the expression of at least one target DNA sequence in a cell, tissue, cellular nucleus, and/or sample comprising chromatin and/or biological effect of specifically epigenetically modifying chromatin in a cell, tissue, cellular nucleus, and/or sample comprising chromatin, if the modulation and/or biological effect in the presence of the agent differs from the modulation and/or biological effect in the absence of the agent or to a control.

15. A method for the prevention and/or treatment of a disease, wherein said method comprises administering to a subject in need of such prevention or treatment at least one complex according to claim 1.

16. The complex according to claim 1, wherein the catalytically inactive site-specific nuclease is selected from the group consisting of a catalytically dead (d) Cas9 from Streptococcus pyogenes, asCas12, saCas9, miniCas9, dCas9, fCas9, SceI, and dCas9/fCas9 fusions.

17. The complex according to claim 1, wherein said specific chromatin modifying activity is selected from a specific DNA methylation activity, a histone methylation activity, a specific histone acetylation or ubiquitination activity, and a specific chromatin demethylation/deacetylation activity.

18. The complex according to claim 2, wherein the linker sequence is Streptococcus pyogenes dCas9GCN4₍₃₋₇₎.

19. The complex according to claim 1, wherein the linker sequence comprises glycine (G) and serine (S) amino acids.

20. The method according to claim 15, used for the prevention and/or treatment of genetic disorders, proliferative disorders, immune cells that produce autoantibodies, bacterial or viral infections, protozoan infections, fragile-X syndrome, muscular dystrophy, kidney injury, cardiovascular diseases, shortened organismal lifespan, tissue aging, neurodegenerative disorders, Alzheimer's disease (AD), Parkinson's disease (PD), Huntington's disease (HD), Amyotrophic lateral sclerosis (ALS), alcohol abuse disorder, and/or epigenetic diseases.
